(19) United States
(12) Patent Application Publication
(10) Pub. No.: US 2019/0100732 A1
(43) Pub. Date: Apr. 4, 2019

(54) ASSAY FOR THE REMOVAL OF METHYL-CYTOSINE RESIDUES FROM DNA

(71) Applicant: Yissum Research Development Company of the Hebrew University of Jerusalem Ltd., Jerusalem (IL)

(72) Inventors: Asaf HELLMAN, Kibbutz Yotvata (IL); Nurit MERON, Ramat-Gan (IL)

(73) Assignee: Yissum Research Development Company of the Hebrew University of Jerusalem Ltd., Jerusalem (IL)

(21) Appl. No.: 16/086,616

(22) PCT Filed: Jun. 2, 2017
(86) PCT No.: PCT/IL2017/050617

§ 371 Date: Sep. 20, 2018

Related U.S. Application Data
(60) Provisional application No. 62/344,494, filed on Jun. 2, 2016.

(51) Int. Cl. C12N 9/02; C12N 15/113; C12N 15/62; C12N 9/22; C12N 15/10

(52) U.S. Cl. CPC C12N 9/0071; C12N 15/113; C12N 15/62; C12N 9/22; C12N 2310/20; C12Y 114/11; C07K 2319/01; C07K 2319/80; C07K 2319/85; C12N 15/102

(57) ABSTRACT

An isolated polynucleotide encoding a fusion protein which comprises a catalytically inactive CRISPR associated 9 (dCas9) protein linked to a TET protein is disclosed. Use thereof and of the fusion protein itself is also disclosed.

### FIELD AND BACKGROUND OF THE INVENTION

The present invention, in some embodiments thereof, relates to nucleic acid sequences which encode fusion proteins which modify methylation of a target gene and to fusion proteins that modify the methylation of a target gene.

The recent emergence of approaches that allow tailored editing of the epigenome has been possible in part due to enormous advances in genetic engineering. A common feature of new epigenetic tools is that they employ unique DNA sequences as a molecular homing device for secondary effector proteins that are capable of robust epigenetic reorganization. At the forefront of these approaches are tools built upon the nucleotide sequence recognition capacities native to three different systems: zinc-finger nucleases (ZFNs), transcriptional-activator like effectors (TALEs), and clustered regularly interspaced short palindromic repeats (CRISPR), which interact with Cas9 nucleases. Although these simple biochemical systems evolved for very different purposes, each employ an innate ability to recognize and bind specific DNA sequences, and each can be readily re-engineered to utilize this capacity for interrogation of the epigenome.

CRISPR/Cas approaches were first discovered in bacteria, where they serve as a form of adaptive immune defense against viruses and plasmids. However, CRISPR tools use engineered “guide” RNA (gRNA), which is a synthetic combination of two separate small RNAs endogenous to the bacterial system. These gRNAs have the dual function of binding specific regions of DNA (they can be engineered to bind to almost any site in DNA), and serving as a scaffold to recruit CRISPR associated proteins to DNA (such as the nuclease Cas9). Moreover, Cas9 can be modified such that it has no nuclease activity, but retains its gRNA binding capabilities.

In their simplest form, synthetic CRISPR gRNAs are used to direct cleavage of specific sequences of DNA, which is highly useful for deletion of genetic material in genome engineering. However, almost simultaneously with the emergence of these techniques, many groups realized that the basic DNA binding capabilities of these tools could also be used to target fused effector proteins to DNA. Thus, beyond its ability to cut or nick double-stranded DNA, CRISPR approaches can ferry other cargo to DNA, including transcription factors, generic transcriptional activators, and transcriptional repressors. These tools therefore enable relatively straightforward yet highly robust interrogation of the functional roles of specific genes and gene products.

DNA methylation, an epigenetic process by addition of a methyl group to DNA, mainly occurs at the fifth carbon of cytosine base within CpG dinucleotide. In mammalian cells, DNA methylation regulates gene expression and thus has critical roles in a myriad of physiological and pathological processes, which include, but are not limited to, cell development and differentiation, genome imprinting and tumorigenesis.

Thus, targeting of DNA methylation enzymes to specific DNA sequences with TALE or CRISPR-based tools has the potential to revolutionize our understanding of the functional consequences of DNA methylation and demethylation. A general proof-of-concept for this approach has already been demonstrated using several targeting strategies. For example, targeting of the mammalian DNA methyltransferases Dnrnt3a directly to the MASPIN or SOX2 genes in breast cancer cell lines led to stable increases in DNA methylation at these genes, which were heritable across cell division and associated with robust gene repression (Rivenbark AG., et al., Epigenetics. 2012; 7:350-360).

Likewise, demethylation of specific nucleotides in human cells has been accomplished by fusing the catalytic domain of the Tetl enzyme to a custom TALE array targeting several genes individually (Maeder ML., Nat Biotechnol. 2013; 31:1137-1142).

Finally, targeted DNA demethylation has also been accomplished by fusing thymine deglycosylase (TDG) to the DNA binding domain of a transcription factor. Gregory DJ., et al., Epigenetics. 2012; 7:344-349.

Vojta et al (Nucleic Acid Research 2016 doi: 10.1093/nar/gkw159) teach CRISPR guided methylation of DNA.

Additional art includes Xu et al., Cell Discovery 2, 2016, doi: 10.1038/celldisc.2016.9 and US Patent Application No. 20160010076.

### SUMMARY OF THE INVENTION

According to an aspect of some embodiments of the present invention there is provided an isolated polynucleotide encoding a fusion protein which comprises a catalytically inactive CRISPR associated 9 (dCas9) protein linked to a TET protein. According to an aspect of some embodiments of the present invention there is provided a polypeptide comprising catalytically inactive CRISPR associated 9 (dCas9) protein linked to a TET protein.

According to an aspect of some embodiments of the present invention there is provided an expression vector comprising the described herein.

According to an aspect of some embodiments of the present invention there is provided a cell which expresses the polynucleotide described herein.

According to an aspect of some embodiments of the present invention there is provided a kit comprising the polynucleotide described herein and at least one guide RNA which is directed to a predetermined target gene.

According to an aspect of some embodiments of the present invention there is provided a kit comprising the polynucleotide described herein and a polynucleotide that encodes a fusion protein comprising catalytically inactive CRISPR associated 9 (dCas9) protein linked to an enzyme selected from the group consisting of DNA methyltransferase (DNMT), histone acetyltransferase (HAT), histone deacetylase (HDAC), histone methyltransferase (HMT) and histone demethylase.

According to an aspect of some embodiments of the present invention there is provided a method of modifying DNA methylation of a target gene in a cell, the method comprising expressing the polynucleotide described herein in the cell, and one or more guide RNA directed to the target gene.

According to embodiments of the present invention, the TET protein is TET1.

According to embodiments of the present invention, the TET1 is human TET1.

According to embodiments of the present invention, the TET protein comprises the catalytic domain of the TET protein.

According to embodiments of the present invention, the fusion protein comprises a single copy of the TET protein.

According to embodiments of the present invention, the catalytic domain of the TET protein comprises a sequence as least 90% identical to the sequence as set forth in SEQ ID NO: 1.

According to embodiments of the present invention, the catalytic domain of the TET protein comprises a sequence 100% identical to the sequence as set forth in SEQ ID NO: 1.

According to embodiments of the present invention, the catalytic domain is linked directly to the dCas9.

According to embodiments of the present invention, the catalytic domain is linked to the dCas9 via a peptide linker.

According to embodiments of the present invention, the peptide linker comprises the sequence as set forth in SEQ ID NO: 3 (Gly, Gly, Gly, Gly, Ser).

According to embodiments of the present invention, the catalytically inactive Cas9 protein comprises mutations at a site selected from the group consisting of D10, E762, H983, D986, H840 and N863.

According to embodiments of the present invention, the mutations are: (i) D10A or D10N, and (ii) H840A, H840N, or H840Y.

According to embodiments of the present invention, the mutations are D10A and H840A.

According to embodiments of the present invention, the dCAS9 comprises the sequence as set forth in SEQ ID NO: 2.

According to embodiments of the present invention, the TET protein is linked to the C terminus of the dCas9.

According to embodiments of the present invention, the TET protein is linked to the N terminus of the dCas9.

According to embodiments of the present invention, the fusion protein comprises an amino acid sequence as set forth in SEQ ID NO: 4.

According to embodiments of the present invention, the isolated polynucleotide comprises a nucleic acid sequence as set forth in SEQ ID NO: 15.

According to embodiments of the present invention, the cell is a stem cell.

According to embodiments of the present invention, the stem cell is a mesenchymal stem cell, an embryonic stem cell or an induced pluripotent stem cell.

According to embodiments of the present invention, the cell is a cancer cell.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1: Exemplary design of the fusion proteins. Human TET catalytic domain fused to dCas9. The domain sequence is 100% identical to TET1 protein and shared 61% identity with TET2 and 54% with TET3. Point mutations in the Fe(II)-binding sites inactivated demethylation but maintain the targeting capability. CXXC: zinc-binding domain. CD: Cys-rich domain. DSBH: Double-stranded β-helix 20G-Fe(II)-dependent dioxygenase domain. Gray lines: Fe(II)-binding sites. Red lines: 2-Oxoglutarate-binding site.

FIG. 2: Delivery of the gRNA to cells. A. general structure of the gRNA, consisting of the target sequence (N(20)) and the gRNA scaffold. B. Map of a typical gRNA expression vector.

FIG. 3A: structure of exemplary gRNA that can be used for the present invention (SEQ ID NO: 61).

FIG. 3B: Map of the dCas9:TET expression vector.

FIGS. 4A-B. Targeted demethylation using dcas9-TET fusion in KCNE4 A. Schematic illustrating the human KCNE4 locus in chromosome 2 with CpG island within the gene. Two sgRNAs (red arrows) were used to direct the dCas9-TET fusion protein to a region within the CpG island. The CpG position within KCNE4 gene is indicated by the distance from KCNE4 TSS and within CpG island the coordinates indicate the position in chromosome 2. The sequence region is marked within the CpG island in orange and the region with the most significant effect is marked in bold. B. DNA methylation levels resulted from targeting experiments with the dCas9-TET fusion protein (TET), or with the dCas9-TET inactive fusion protein (TET inactive), guided by sgRNAs 7 and 8 and cells without transfection (control). Each experiment included three independent samples of bisulfite PCR amplification followed by high-throughput next-generation sequencing. The difference in methylation in each site was calculated by difference between the average methylation in TET inactive samples and the average methylation in TET sample.

FIG. 5 is a graph illustrating the time course of targeted demethylation effect The methylation level at represented CpG site with the most significant effect after 7 days (chr2: 223,917,805). Means of methylation of three independent samples are shown with bars representing statistical deviation.

FIGS. 6A-B illustrate the targeted demethylation at specific CpG site in HBB promoter. A. The human HBB locus with CpGs indicated with black arrows. Numbering indicates position on the DNA relative to the start site of transcription (right-angle arrow). Colored arrows indicate the location and direction (5′ to 3′) of sgRNA. B. DNA methylation levels resulted from targeting experiments with the dCas9-TET fusion protein (TET), or with the dCas9-TET inactive fusion protein (TET inactive), guided by three sgRNAs and cells without transfection (control) or with transfection with GFP expressing vector only. The coordinates of the CpG sites in chromosome 11 are indicated in the first row of the table. The experiments with TET active or TET inactive included three independent samples of bisulfite PCR amplification followed by high-throughput next-generation sequencing.

FIG. 7 is a graph illustrating the reactivation of HBB expression following specific targeted DNA demethylation. Expression levels of the endogenous HBB gene after targeting dcas9:TET or dcas9:TET inactive relative to cells without transfection. Results of average of two independent biologic repeats are shown with error bars representing standard deviation.

FIG. 8 is a graph illustrating the downregulation of SPI1 expression following targeted mutations in PU.1 enhancer. Expression levels of the endogenous SPIT gene after targeted mutations in the enhancer, relative to cells without transfection. Results of an average of three independent biologic repeats are shown with error bars representing standard deviation.

FIG. 9 is a graph illustrating the expression levels of VEGFA following mutation in the VEGFA enhancer. Expression levels of the endogenous VEGFA gene in the mutated clones relative to mock-treated cell. Results of three independent qPCR experiments with three technical replications of each experiment are shown. The error bars represent standard deviation.

FIG. 10 illustrates the DNA methylation levels resulting from targeting cas9 to PU.1 enhancer in clones of k562 cells. The methylation levels in 8 CpG sites in the sgRNA region were evaluated by bisulfite followed by next-generation sequencing in two clones compare to control cells-untransfected cells. The first row shows the coordinates of the examined CpG sites in chromosome 11. The last row shows the difference in methylation levels between the average in CRISPR/Cas9 clones and the control cells.

### DESCRIPTION OF SPECIFIC EMBODIMENTS OF THE INVENTION

The present invention, in some embodiments thereof, relates to nucleic acid sequences which encode fusion proteins which modify methylation of a target gene.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

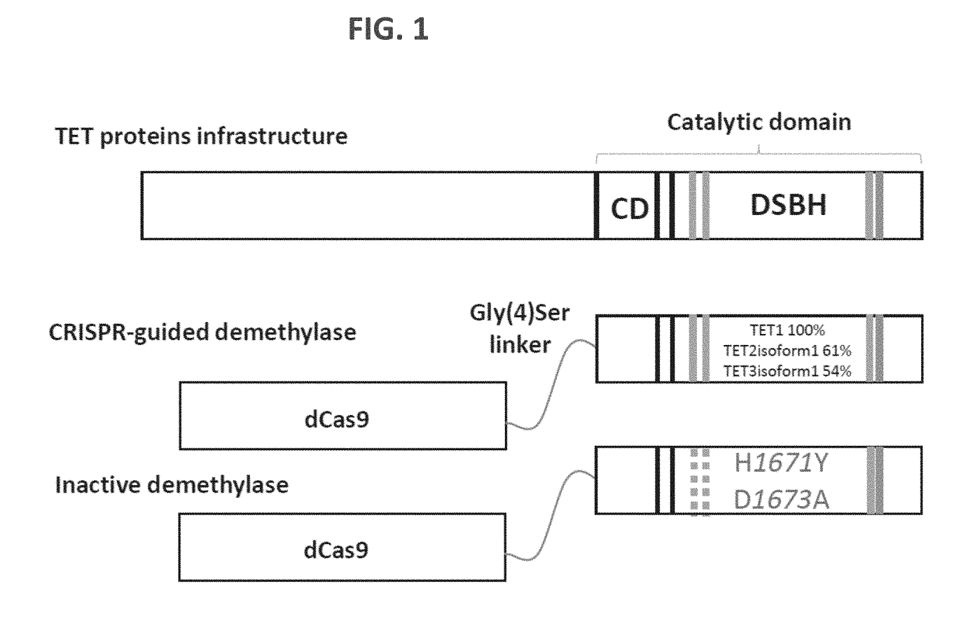

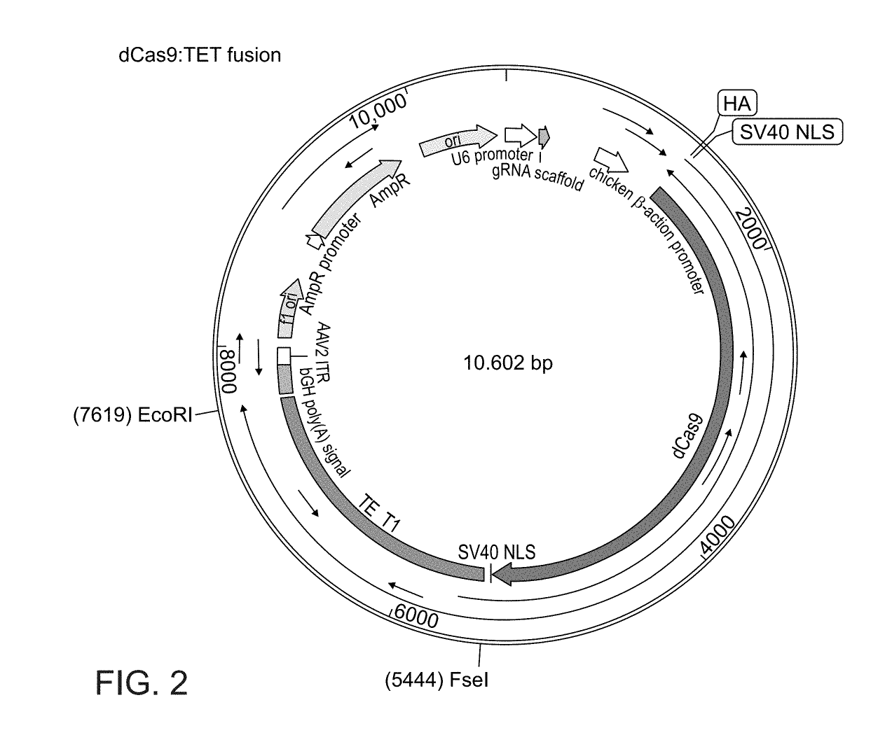

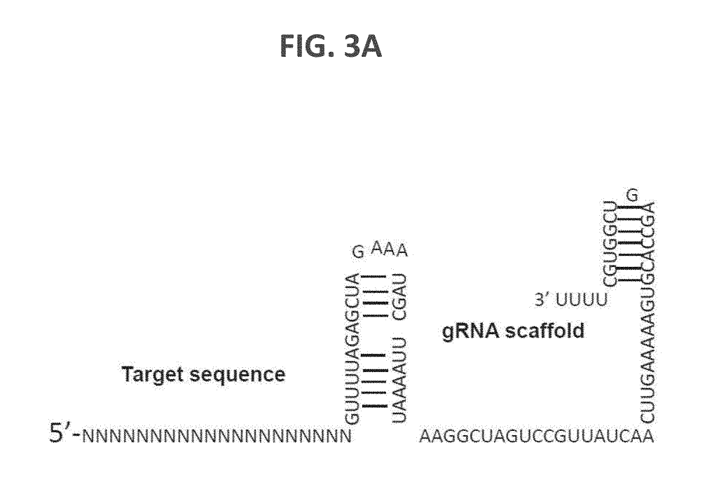

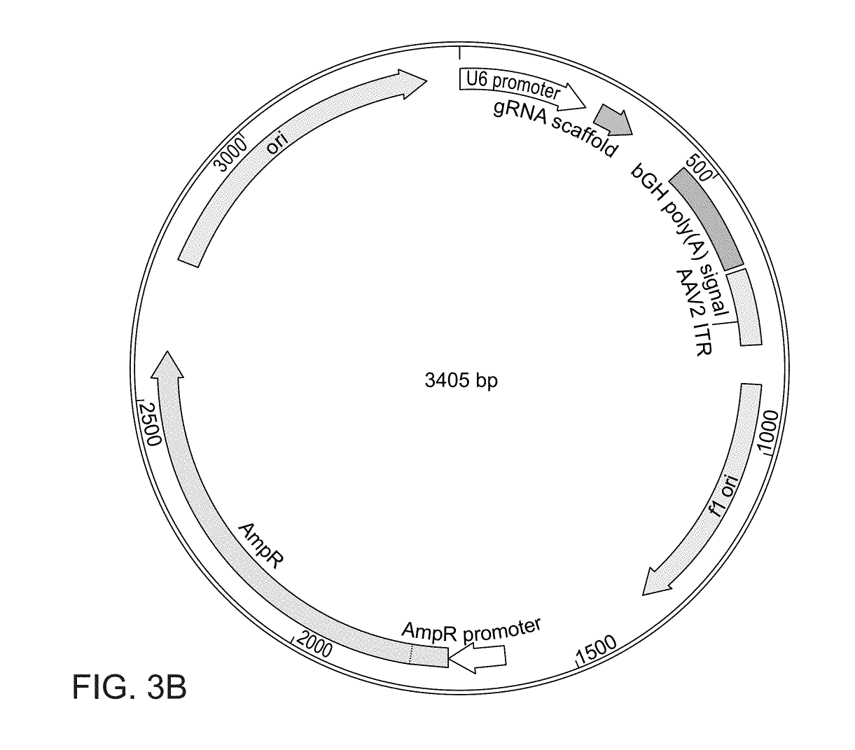

The present inventors have conceived of a new approach for efficient targeting of demethylation based on CRISPR technology. The new epigenetics editing system consist of mutated endonuclease Cas9 (dCas9) protein fused to the demethylation catalytic domain (dCas9:TET). The DNA coding sequence of the TET catalytic domains was integrated contiguously to the dCas9 coding sequence in a modified vector backbone obtained from an open resource. A short flexible linker made of four glycine and one serine amino acids was placed between the fused protein domains to eliminate interference (FIG. 1).

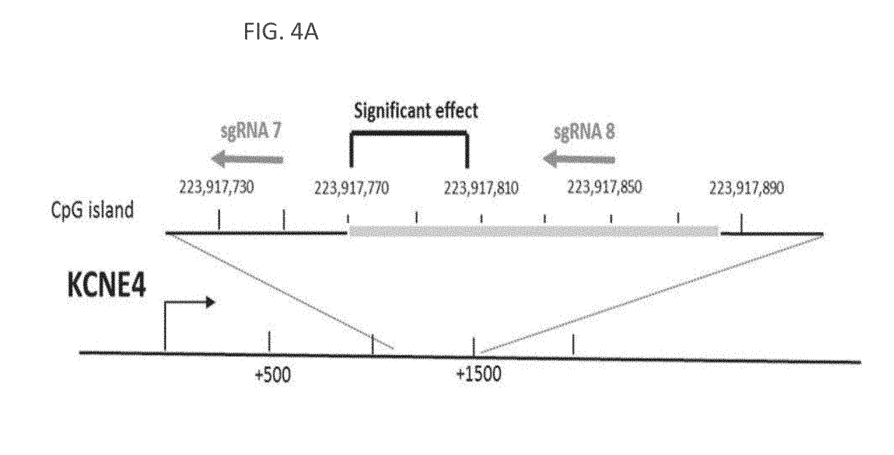

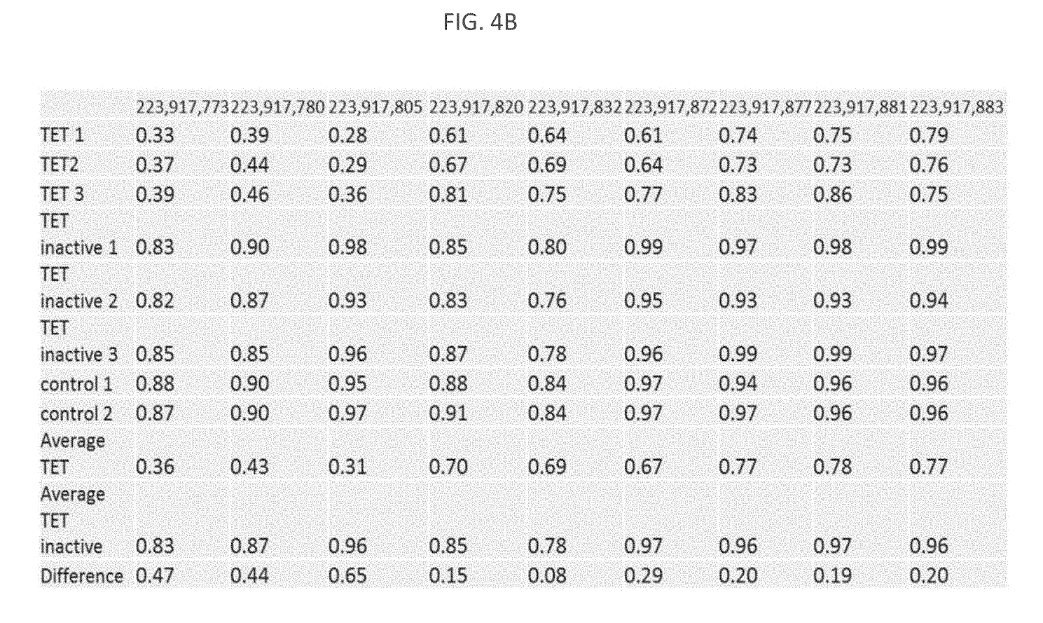

The fusion of dCas9:TET induced significant demethylation at the targeted KCNE4 gene region. The maximal observed effect was 44-65 reduced methylation percentages in 3 CpG sites located 18-50 base pairs downstream to the PAM sequence, 7 days post-transfection (FIGS. 4A-B). Importantly, demethylation occurred in spite of the expression of de-novo DNA methyltransferases (DNMT3A, DNMT3B), a hallmark of many cancers.

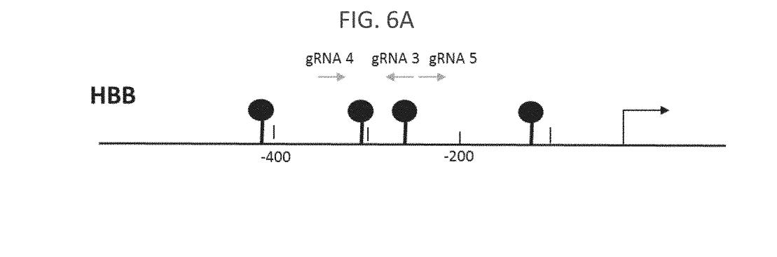

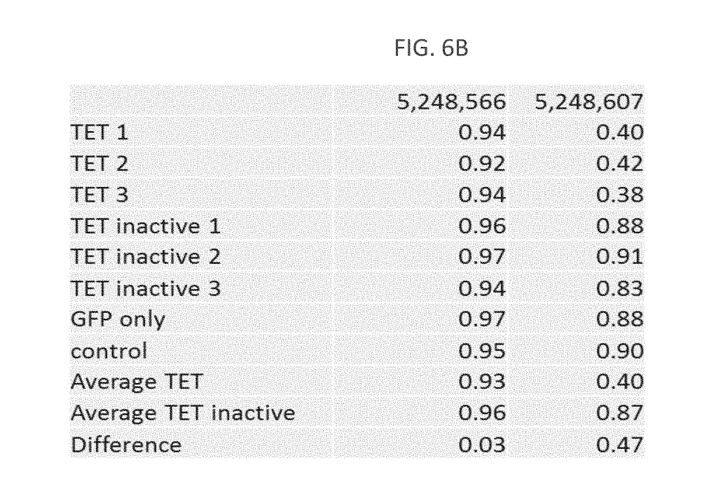

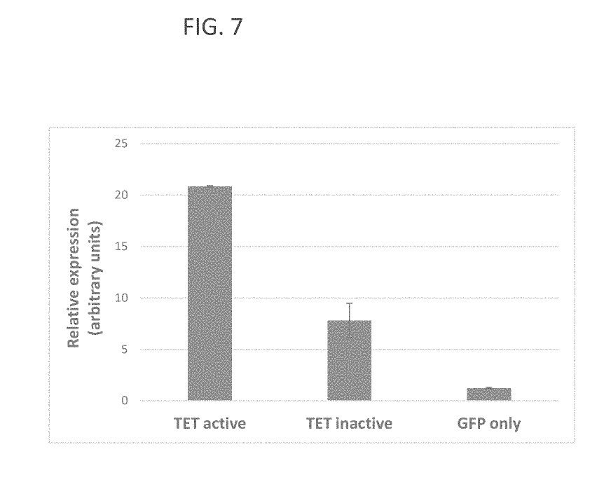

Whilst further reducing the present invention to practice, the present inventors showed that a demethylation of about 47% at a single CpG site in HBB promoter was sufficient for increasing HBB gene expression (FIGS. 6B and 7). The dynamic of de-methylation re-methylation processes was also investigated in living cells. Seven days following targeted demethylation of the KCNE4 CpG island, methylation levels gradually recovered at the examined CpG sites. Thus, expression of the fusion dcas9:TET was shown to be sufficient to induce demethylation even in the presence of DNMTs, but upon removal, the low methylation at the regulatory sites was not maintained.

Thus, according to a first aspect of the present invention there is provided polypeptide comprising catalytically inactive CRISPR associated 9 (dCas9) protein linked to a TET protein.

Cas9

Cas9 molecules of a variety of species can be used in the methods and compositions described herein. While the S. pyogenes and S. thermophilus Cas9 molecules are exemplified herein, Cas9 molecules of, derived from, or based on the Cas9 proteins of other species listed in US Patent Application No. 20160010076 can be used as well. Additional Cas9 proteins are described in Esvelt et al., Nat Methods. 2013 November; 10(11):1116-21 and Fonfara et al., “Phylogeny of Cas9 determines functional exchangeability of dual-RNA and Cas9 among orthologous type II CRISPR-Cas systems.” Nucleic Acids Res. 2013 Nov. 22. doi:10.1093/nar/gkt1074.

The constructs and methods described herein can include the use of any of those Cas9 proteins, and their corresponding guide RNAs or other guide RNAs that are compatible. The Cas9 from Streptococcus thermophilus LMD-9 CRISPR1 system has been shown to function in human cells in Cong et al (Science 339, 819 (2013)). Additionally, Jinek et al. showed in vitro that Cas9 orthologs from S. thermophilus and L. innocua, can be guided by a dual S. pyogenes gRNA to cleave target plasmid DNA, albeit with slightly decreased efficiency.

In some embodiments, the present system utilizes the Cas9 protein from S. pyogenes, either as encoded in bacteria or codon-optimized for expression in mammalian cells (e.g. human cells), containing mutations at D10, E762, H983, or D986 and H840 or N863, e.g., D10A/D10N and H840A/H840N/H840Y, to render the nuclease portion of the protein catalytically inactive; substitutions at these positions could be alanine (as they are in Nishimasu al., Cell 156, 935-949 (2014)) or they could be other residues, e.g., glutamine, asparagine, tyrosine, serine, or aspartate, e.g., E762Q, H983N, H983Y, D986N, N863D, N863S, or N863H. The sequence of the catalytically inactive S. pyogenes Cas9 that can be used in the methods and compositions described herein is as set forth in SEQ ID NO: 2.

In some embodiments, the Cas9 nuclease used herein is at least about 50% identical to the sequence of S. pyogenes Cas9, i.e., at least 50% identical to SEQ ID NO: 2. In some embodiments, the nucleotide sequences are about 50%, 55%, 60%, 65%, 70%, 75%, 80%, 85%, 90%, 95%, 99% or 100% identical to SEQ ID NO: 2.

In some embodiments, the catalytically inactive Cas9 used herein is at least about 50% identical to the sequence of the catalytically inactive S. pyogenes Cas9, i.e., at least 50%, 55%, 60%, 65%, 70%, 75%, 80%, 85%, 90%, 95%, 99% or 100% identical to SEQ ID NO:2, wherein the mutations at D10 and H840, e.g., D10A/D10N and H840A/H840N/H840Y are maintained.

In some embodiments, any differences from SEQ ID NO:2 are in non-conserved regions, as identified by sequence alignment of sequences set forth in Chylinski et al., RNA Biology 10:5, 1-12; 2013; Esvelt et al., Nat Methods. 2013 November; 10(11):1116-21 and Fonfara et al., Nucl. Acids Res. (2014) 42 (4): 2577-2590, and wherein the mutations at D10 and H840, e.g., D10A/D10N and H840A/H840N/H840Y are maintained.

To determine the percent identity of two sequences, the sequences are aligned for optimal comparison purposes (gaps are introduced in one or both of a first and a second amino acid or nucleic acid sequence as required for optimal alignment, and non-homologous sequences can be disregarded for comparison purposes). The length of a reference sequence aligned for comparison purposes is at least 50% (in some embodiments, about 50%, 55%, 60%, 65%, 70%, 75%, 85%, 90%, 95%, or 100% of the length of the reference sequence) is aligned. The nucleotides or residues at corresponding positions are then compared. When a position in the first sequence is occupied by the same nucleotide or residue as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. For purposes of the present application, the percent identity between two amino acid sequences is determined using the Needleman and Wunsch ((1970) J. Mol. Biol. 48:444-453) algorithm which has been incorporated into the GAP program in the GCG software package, using a Blossum 62 scoring matrix with a gap penalty of 12, a gap extend penalty of 4, and a frameshift gap penalty of 5.

An exemplary nucleic acid sequence which can be used to express Cas9 nuclease is set forth in SEQ ID NO: 5. The sequence may be at least 90%, 91%, 92%, 93%, 94%, 95%, 96%, 97%, 98%, 99% or 100% homologous or identical to SEQ ID NO: 5.

TET Protein

The TET protein can be fused on the N or C terminus of the Cas9. Sequences for human TET1-3 are known in the art, examples of which are listed in US Patent Application No. 20160010076. In some embodiments, all or part of the full-length sequence of the catalytic domain of the TET protein can be included, e.g., the Tet1 catalytic domain comprising amino acids 1580-2052, Tet2 comprising amino acids 1290-1905 and Tet3 comprising amino acids 966-1678. See, e.g., FIG. 1 of Iyer et al., Cell Cycle. 2009 Jun. 1; 8(11):1698-710. Epub 2009 Jun. 27, for an alignment illustrating the key catalytic residues in all three Tet proteins, and the supplementary materials thereof (available at ftp site ftp(dot)ncbi(dot)nih(dot)gov/pub/aravind/DONS/supplementary_material_DONS(dot)ht ml) for full length sequences; in some embodiments, the sequence includes amino acids 1418-2136 of Tet1 or the corresponding region in Tet2/3.

According to a particular embodiment, the amino acid sequence of the TET protein is human TET 1 protein (NCBI Reference Sequence: NP_085128.2) as set forth in SEQ ID NO: 1, or is at least 90%, 91%, 92%, 93%, 94%, 95%, 96%, 97%, 98%, 99% or 100% identical to the sequence as set forth in SEQ ID NO: 1. An exemplary nucleic acid sequence which encodes human TET 1 protein is set forth in SEQ ID NO: 6.

In one embodiment, the human TET protein comprises the catalytic domain only. Thus, in the case of TET1, the protein has a sequence as set forth in SEQ ID NO: 7, or is at least 90%, 91%, 92%, 93%, 94%, 95%, 96%, 97%, 98%, 99% or 100% identical to the sequence as set forth in SEQ ID NO: 7. An exemplary nucleic acid sequence which encodes human TET 1 protein catalytic domain is set forth in SEQ ID NO: 8.

According to a particular embodiment, the amino acid sequence of the TET protein is human TET 2 protein (NCBI Reference Sequence: NM_001127208.2) as set forth in SEQ ID NO: 9, or is at least 90%, 91%, 92%, 93%, 94%, 95%, 96%, 97%, 98%, 99% or 100% identical to the sequence as set forth in SEQ ID NO: 9. An exemplary nucleic acid sequence which encodes human TET 2 protein is set forth in SEQ ID NO: 10.

According to a particular embodiment, the amino acid sequence of the TET protein is human TET 3 protein (NCBI Reference Sequence: NM_001127208.2) as set forth in SEQ ID NO: 11, or is at least 90%, 91%, 92%, 93%, 94%, 95%, 96%, 97%, 98%, 99% or 100% identical to the sequence as set forth in SEQ ID NO: 11. An exemplary nucleic acid sequence which encodes human TET 3 protein is set forth in SEQ ID NO: 12.

In some embodiments, the fusion proteins include a linker between the dCas9 and the TET protein. Linkers that can be used in these fusion proteins (or between fusion proteins in a concatenated structure) can include any sequence that does not interfere with the function of the fusion proteins. In preferred embodiments, the linkers are short, e.g., 2-20 amino acids, and are typically flexible (i.e., comprising amino acids with a high degree of freedom such as glycine, alanine, and serine). In some embodiments, the linker comprises one or more units consisting of GGGS (SEQ ID NO:13) or GGGGS (SEQ ID NO:3), e.g., two, three, four, or more repeats of the GGGS (SEQ ID NO:13) or GGGGS (SEQ ID NO:3) unit. Other linker sequences can also be used.

Expression Systems:

In order to use the fusion proteins described herein, it may be desirable to express them from a nucleic acid that encodes them.

Thus, according to another aspect of the present invention there is provided an isolated polynucleotide encoding a fusion protein which comprises a catalytically inactive CRISPR associated 9 (dCas9) protein linked to a TET protein.

As used herein the term “polynucleotide” refers to a single or double stranded nucleic acid sequence which is isolated and provided in the form of an RNA sequence, a complementary polynucleotide sequence (cDNA), a genomic polynucleotide sequence and/or a composite polynucleotide sequences (e.g., a combination of the above).

The polynucleotide of this aspect of the present invention may encode a single copy of the TET protein or multiple copies of the TET protein.

An exemplary nucleic acid sequence encoding the fusion protein of this aspect of the present invention is set forth in SEQ ID NO: 15.

Expression from the polynucleotide of this aspect of the present invention can be performed in a variety of ways. For example, a nucleic acid encoding a fusion protein can be cloned into an intermediate vector for transformation into prokaryotic or eukaryotic cells for replication and/or expression. Intermediate vectors are typically prokaryote vectors, e.g., plasmids, or shuttle vectors, or insect vectors, for storage or manipulation of the nucleic acid encoding the fusion protein or for production of the fusion protein. The nucleic acid encoding the fusion protein can also be cloned into an expression vector, for administration to a plant cell, animal cell, preferably a mammalian cell (e.g. a human cell), fungal cell, bacterial cell, or protozoan cell.

To bring about expression, a sequence encoding the fusion protein is typically subcloned into an expression vector that contains a promoter to direct transcription. Suitable bacterial and eukaryotic promoters are well known in the art and described, e.g., in Sambrook et al., Molecular Cloning, A Laboratory Manual (3d ed. 2001); Kriegler, Gene Transfer and Expression: A Laboratory Manual (1990); and Current Protocols in Molecular Biology (Ausubel et al., eds., 2010). Bacterial expression systems for expressing the engineered protein are available in, e.g., E. coli, Bacillus sp., and Salmonella (Palva et al., 1983, Gene 22:229-235). Kits for such expression systems are commercially available. Eukaryotic expression systems for mammalian cells, yeast, and insect cells are well known in the art and are also commercially available.

The promoter used to direct expression of the nucleic acid depends on the particular application. For example, a strong constitutive promoter is typically used for expression and purification of fusion proteins. In contrast, when the fusion protein is to be administered in vivo for gene regulation, either a constitutive or an inducible promoter can be used, depending on the particular use of the fusion protein. In addition, a preferred promoter for administration of the fusion protein can be a weak promoter, such as HSV TK or a promoter having similar activity. The promoter can also include elements that are responsive to transactivation, e.g., hypoxia response elements, Ga14 response elements, lac repressor response element, and small molecule control systems such as tetracycline-regulated systems and the RU-486 system (see, e.g., Gossen & Bujard, 1992, Proc. Natl. Acad. Sci. USA, 89:5547; Oligino et al., 1998, Gene Ther., 5:491-496; Wang et al., 1997, Gene Ther., 4:432-441; Neering et al., 1996, Blood, 88:1147-55; and Rendahl et al., 1998, Nat. Biotechnol., 16:757-761).

In addition to the promoter, the expression vector typically contains a transcription unit or expression cassette that contains all the additional elements required for the expression of the nucleic acid in host cells, either prokaryotic or eukaryotic. A typical expression cassette thus contains a promoter operably linked, e.g., to the nucleic acid sequence encoding the fusion protein, and any signals required, e.g., for efficient polyadenylation of the transcript, transcriptional termination, ribosome binding sites, or translation termination. Additional elements of the cassette may include, e.g., enhancers, and heterologous spliced intronic signals.

The particular expression vector used to transport the genetic information into the cell is selected with regard to the intended use of the fusion protein, e.g., expression in plants, animals, bacteria, fungus, protozoa, etc. Standard bacterial expression vectors include plasmids such as pBR322 based plasmids, pSKF, pET23D, and commercially available tag-fusion expression systems such as GST and LacZ. A preferred tag-fusion protein is the maltose binding protein (MBP). Such tag-fusion proteins can be used for purification of the engineered protein. Epitope tags can also be added to recombinant proteins to provide convenient methods of isolation, for monitoring expression, and for monitoring cellular and subcellular localization, e.g., c-myc or FLAG.

Expression vectors containing regulatory elements from eukaryotic viruses are often used in eukaryotic expression vectors, e.g., SV40 vectors, papilloma virus vectors, and vectors derived from Epstein-Barr virus. Other exemplary eukaryotic vectors include PMSG, pAV009/A+, pMTO10/A+, pMAMneo-5, baculovirus pDSVE, and any other vector allowing expression of proteins under the direction of the SV40 early promoter, SV40 late promoter, metallothionein promoter, murine mammary tumor virus promoter, Rous sarcoma virus promoter, polyhedrin promoter, or other promoters shown effective for expression in eukaryotic cells. Some expression systems have markers for selection of stably transfected cell lines such as thymidine kinase, hygromycin B phosphotransferase, and dihydrofolate reductase. High yield expression systems are also suitable, such as using a baculovirus vector in insect cells, with the fusion protein encoding sequence under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of recombinant sequences.

Standard transfection methods are used to produce bacterial, mammalian, yeast or insect cell lines that express large quantities of protein, which are then purified using standard techniques (see, e.g., Colley et al., 1989, J. Biol. Chem., 264:17619-22; Guide to Protein Purification, in Methods in Enzymology, vol. 182 (Deutscher, ed., 1990)). Transformation of eukaryotic and prokaryotic cells are performed according to standard techniques (see, e.g., Morrison, 1977, J. Bacteriol. 132:349-351; Clark-Curtiss & Curtiss, Methods in Enzymology 101:347-362 (Wu et al., eds, 1983).

Any of the known procedures for introducing foreign nucleotide sequences into host cells may be used. These include the use of calcium phosphate transfection, polybrene, protoplast fusion, electroporation, nucleofection, liposomes, microinjection, naked DNA, plasmid vectors, viral vectors, both episomal and integrative, and any of the other well-known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing the protein of choice.

In some embodiments, the fusion protein includes a nuclear localization domain which provides for the protein to be translocated to the nucleus. Several nuclear localization sequences (NLS) are known, and any suitable NLS can be used. For example, many NLSs have a plurality of basic amino acids, referred to as a bipartite basic repeats (reviewed in Garcia-Bustos et al, 1991, Biochim. Biophys. Acta, 1071:83-101). An NLS containing bipartite basic repeats can be placed in any portion of chimeric protein and results in the chimeric protein being localized inside the nucleus. In preferred embodiments a nuclear localization domain is incorporated into the final fusion protein, as the ultimate functions of the fusion proteins described herein will typically require the proteins to be localized in the nucleus.

An exemplary NLS is provide in SEQ ID NO: 14.

The expression construct may comprise 1, 2, 3 or more NLS.

The polynucleotide of this aspect of the present invention may be provided per se or may be part of a kit for modifying DNA methylation.

The kit may comprise guide RNAs (gRNAs) that target to a gene of interest. The kit may comprise a plurality of gRNAs that target a single gene of interest. Alternatively, the kit may comprise a plurality of gRNAs that target several genes of interest. The gRNA may target any part of a gene—for example the coding region, the promoter region, an enhancer region etc.

In one embodiment, one strand of the DNA is targeted. In another embodiment, both strands of the DNA may be used simultaneously as targets to multiple gRNAs.

The target site may be selected such that expression of the endogenous gene is altered. Expression of the endogenous gene may be increased or decreased using this method. In one embodiment, the gRNA targets the VEGFA gene. In another embodiment, the gRNA targets the beta globin gene.

Guide RNAs (gRNAs)

Guide RNAs generally speaking come in two different systems: System 1, which uses separate crRNA and tracrRNAs that function together to guide cleavage by Cas9, and System 2, which uses a chimeric crRNA-tracrRNA hybrid that combines the two separate guide RNAs in a single system (referred to as a single guide RNA or sgRNA, see also Jinek et al., Science 2012; 337:816-821). The tracrRNA can be variably truncated and a range of lengths has been shown to function in both the separate system (system 1) and the chimeric gRNA system (system 2). For example, in some embodiments, tracrRNA may be truncated from its 3′ end by at least 1, 2, 3, 4, 5, 6, 7, 8, 9, 10, 15, 20, 25, 30, 35 or 40 nts. In some embodiments, the tracrRNA molecule may be truncated from its 5′ end by at least 1, 2, 3, 4, 5, 6, 7, 8, 9, 10, 15, 20, 25, 30, 35 or 40 nts. Alternatively, the tracrRNA molecule may be truncated from both the 5′ and 3′ end, e.g., by at least 1, 2, 3, 4, 5, 6, 7, 8, 9, 10, 15 or 20 nts on the 5′ end and at least 1, 2, 3, 4, 5, 6, 7, 8, 9, 10, 15, 20, 25, 30, 35 or 40 nts on the 3′ end. See, e.g., Jinek et al., Science 2012; 337:816-821; Mali et al., Science. 2013 Feb. 15; 339(6121):823-6; Cong et al., Science. 2013 Feb. 15; 339(6121):819-23; and Hwang and Fu et al., Nat Biotechnol. 2013 March; 31(3):227-9; Jinek et al., Elife 2, e00471 (2013)). For System 2, generally the longer length chimeric gRNAs have shown greater on-target activity but the relative specificities of the various length gRNAs currently remain undefined and therefore it may be desirable in certain instances to use shorter gRNAs. In some embodiments, the gRNAs are complementary to a region that is within about 100-800 bp upstream of the transcription start site, e.g., is within about 500 bp upstream of the transcription start site, includes the transcription start site, or within about 100-800 bp, e.g., within about 500 bp, downstream of the transcription start site. In some embodiments, vectors (e.g., plasmids) encoding more than one gRNA are used, e.g., plasmids encoding, 2, 3, 4, 5, or more gRNAs directed to different sites in the same region of the target gene.

Cas9 nuclease can be guided to specific 17-20 nt genomic targets bearing an additional proximal protospacer adjacent motif (PAM), e.g., of sequence NGG, using a guide RNA, e.g., a single gRNA or a tracrRNA/crRNA, bearing 17-20 nts at its 5′ end that are complementary to the complementary strand of the genomic DNA target site. Thus, the present methods can include the use of a single guide RNA comprising a crRNA fused to a normally trans-encoded tracrRNA, e.g., a single Cas9 guide RNA as described in Mali et al., Science 2013 Feb. 15; 339(6121):823-6, with a sequence at the 5′ end that is complementary to the target sequence, e.g., of 25-17, optionally 20 or fewer nucleotides (nts), e.g., 20, 19, 18, or 17 nts, preferably 17 or 18 nts, of the complementary strand to a target sequence immediately 5′ of a protospacer adjacent motif (PAM), e.g., NGG, NAG, or NNGG. The guide RNAs can include X._{N} which can be any sequence, wherein N (in the RNA) can be 0-200, e.g., 0-100, 0-50, or 0-20, that does not interfere with the binding of the ribonucleic acid to Cas9.

In some embodiments, the guide RNA includes one or more Adenine (A) or Uracil (U) nucleotides on the 3′ end. In some embodiments the RNA includes one or more U, e.g., 1 to 8 or more Us at the 3′ end of the molecule, as a result of the optional presence of one or more Ts used as a termination signal to terminate RNA PolIII transcription.

Although some of the examples described herein utilize a single gRNA, the methods can also be used with dual gRNAs (e.g., the crRNA and tracrRNA found in naturally occurring systems). In this case, a single tracrRNA would be used in conjunction with multiple different crRNAs expressed using the present system.

In some embodiments, the gRNA is targeted to a site that is at least three or more mismatches different from any sequence in the rest of the genome in order to minimize off-target effects.

Modified RNA oligonucleotides such as locked nucleic acids (LNAs) have been demonstrated to increase the specificity of RNA-DNA hybridization by locking the modified oligonucleotides in a more favorable (stable) conformation. For example, 2′-O-methyl RNA is a modified base where there is an additional covalent linkage between the 2′ oxygen and 4′ carbon which when incorporated into oligonucleotides can improve overall thermal stability and selectivity.

Thus, the gRNAs disclosed herein may comprise one or more modified RNA oligonucleotides. For example, the truncated guide RNAs molecules described herein can have one, some or all of the region of the guideRNA complementary to the target sequence are modified, e.g., locked (2′-O-4′-C methylene bridge), 5′-methylcytidine, 2′-O-methyl-pseudouridine, or in which the ribose phosphate backbone has been replaced by a polyamide chain (peptide nucleic acid), e.g., a synthetic ribonucleic acid.

In other embodiments, one, some or all of the nucleotides of the gRNA sequence may be modified, e.g., locked (2′-O-4′-C methylene bridge), 5′-methylcytidine, 2′-O-methyl-pseudouridine, or in which the ribose phosphate backbone has been replaced by a polyamide chain (peptide nucleic acid), e.g., a synthetic ribonucleic acid.

In some embodiments, the single guide RNAs and/or crRNAs and/or tracrRNAs can include one or more Adenine (A) or Uracil (U) nucleotides on the 3′ end.

The guide RNA may be provided per se or in an expression vector. The vectors for expressing the guide RNAs can include RNA Pol III promoters to drive expression of the guide RNAs, e.g., the H1, U6 or 7SK promoters. These human promoters allow for expression of gRNAs in mammalian cells following plasmid transfection. Alternatively, a T7 promoter may be used, e.g., for in vitro transcription, and the RNA can be transcribed in vitro and purified. Vectors suitable for the expression of short RNAs, e.g., siRNAs, shRNAs, or other small RNAs, can be used.

Deliver or Express the gRNA in the Desire Cells:

The RNA may be delivered to the targeted cells via different methods: First, it is possible to introduce an expression vector with the guide RNA sequence under the appropriate promoter. For this, integrate the templet DNA into an appropriate vector (e.g., addgene #41824), and deliver the vector into the cells using standard transfection protocols as described above. Alternatively, it is possible to introduce PCR amplicon containing gRNA sequence and gRNA scaffold and termination signal under an appropriate promoter (e.g., U6), and deliver it to the cells using one of the above transfection methods. A third possibility is to directly transfect or inject RNA molecules commercially synthesized or produced in the lab. The late methods are preferred when it is needed to simultaneously target many genomic sites in single cells. A selection marker (e.g., antibiotic-resistant gene) can be added to the cells to enrich for transfected cells. The required structure of the gRNA as RNA molecule, PCR amplicon.

As well as gRNAs (or instead of gRNAs), the kit of this aspect of the present invention may comprise at least one additional polynucleotide that encodes a fusion protein comprising catalytically inactive CRISPR associated 9 (dCas9) protein linked to other heterologous functional domains (e.g., transcriptional repressors (e.g., KRAB, ERD, SID, and others, e.g., amino acids 473-530 of the ets2 repressor factor (ERF) repressor domain (ERD), amino acids 1-97 of the KRAB domain of KOX1, or amino acids 1-36 of the Mad mSIN3 interaction domain (SID); see Beerli et al., PNAS USA 95:14628-14633 (1998)) or silencers such as Heterochromatin Protein 1 (HP1, also known as swi6), e.g., HP1.alpha. or HP1.beta.; proteins or peptides that could recruit long non-coding RNAs (lncRNAs) fused to a fixed RNA binding sequence such as those bound by the MS2 coat protein, endoribonuclease Csy4, or the lambda N protein; enzymes that modify histone subunits (e.g., histone acetyltransferases (HAT), histone deacetylases (HDAC), histone methyltransferases (e.g., for methylation of lysine or arginine residues) or histone demethylases (e.g., for demethylation of lysine or arginine residues)) as are known in the art can also be used.

Together with the gRNA, the fusion proteins of the present invention (or polynucleotides encoding same) may be introduced into a wide variety of cell types, embryos at different developmental stages, tissues and species may be targeted, including somatic and embryonic stem cells of human and animal models. In one embodiment, the cell is a stem cell (e.g. a pluripotent stem cell such as an embryonic stem cell or an induced pluripotent stem cell), a mesenchymal stem cell, a tissue stem cell (e.g. a neuronal stem cell or muscle stem cell). In another embodiment, the cell is a healthy cell. In another embodiment, the cell is a diseased cell (e.g, a cancer cell).

In other embodiments the fusion protein (and gRNA) may be injected into the cell. This is particularly relevant for editing of single cells, eggs or embryonic stem cells.

Following introduction of the fusion protein and gRNA described herein, the gene (at the targeted site) may be analyzed to ensure (i.e. confirm) that demethylation has occurred. Thus, for example bisulfite sequencing may be carried out to determine the extent of methylation prior to and/or following the treatment.

Bisulfite sequencing (also known as bisulphite sequencing) is the use of bisulfite treatment of DNA to determine its pattern of methylation.

Treatment of DNA with bisulfite converts cytosine residues to uracil, but leaves 5-methylcytosine residues unaffected. Therefore, DNA that has been treated with bisulfite retains only methylated cytosines. Thus, bisulfite treatment introduces specific changes in the DNA sequence that depend on the methylation status of individual cytosine residues, yielding single-nucleotide resolution information about the methylation status of a segment of DNA. Various analyses can be performed on the altered sequence to retrieve this information. The objective of this analysis is therefore reduced to differentiating between single nucleotide polymorphisms (cytosines and thymidine) resulting from bisulfite conversion.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### Examples

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Methods

The present inventors designed and produced a synthetic protein consisting of mutated endonuclease Cas9 (dCas9) protein fused to the demethylation catalytic domain (dCas9:TET). The DNA coding sequence of the TET catalytic domain was integrated contiguously to the dCas9 coding sequence in modified vector backbone obtained from an open resource. A short linker made of four glycine and one serine amino acids was placed between the fused protein domains to eliminate interference.

A plasmid encoding dCas9 with two inactivating mutations D10A and H840A was obtained from an open resource (Addgene, plasmid #48240), and digested with ECORI and FseI restriction enzymes to remove an unnecessary portion. The human TET1 catalytic domain (amino acids 1418-2136) was amplified from another plasmid (Addgene #49958) using PfuUltra II fusion HS DNA polymerase (Agilent Technologies) with the primers: forward 5′-AGTGGCCGGCCGGAGGCGGTGGAAGCCTGCCCACCTGCAGCTGTC- (SEQ ID NO: 32) 3′ reverse 5′-TCGAATTCTCAGAC CCAATGGTTA-3′ (SEQ ID NO: 33). The amplified product was cloned into p-miniT vector included in a commercial kit (DNA cloning kit, New England Biolabs). Following sequence validation, the catalytic domain was transferred from the cloning vector and integrated into the dCas9 plasmid contiguously to the c-terminus of dCas9 with a gly4ser linker between the two, using a rapid DNA ligation kit (Thermo scientific). The TET catalytic domain with two point mutations (H1671Y and D1673A) was amplified using PfuUltra II fusion HS DNA polymerase (Agilent Technologies) from a TALE-TET1CD plasmid (Addgene #49959) using the same primers as above, cloned as above, sequenced, and ligated into the dCas9 plasmid.

Guide RNA Plasmids:

A human codon-optimized SpCas9 and chimeric gRNA expression plasmid (Addgene #42230) was digested by ECORI and XbaI for cas9 excision, following removal of the staggered ends with Klenow enzyme (NEB), ligation (rapid DNA ligation kit, Thermo scientific) and gel purification. The vector was then digested by BbsI restriction enzyme and gel purified. The phosphorylated oligos (Table 1) were dissolved in DDW at a final concentration of 3 mg/ml and annealed by the following protocol: 1 μl from each oligo were mixed with 48 μl of annealing buffer which composed of 100 mM NaCl (Bio Lab Cat#19032391) and 50 mM Hepes (Biological Industries, Cat#03-025-1C) PH 7.4 in DDW. This reaction was 90° C. for 4 minutes, 70° C. for 10 minutes, 37° C. for 15-20 minutes and 10° C. for 10 minutes. After the annealing, the oligos were ligated to the linearized vector.

TABLE 1
sgRNA sequences targeted to the regulatory
elements of HBB, PU.1 and VEGFA

| | |
|---|---|
| sgRNA PU.1 enhancer | Forward: 5′-CACCGGGCCGGCGCCTGAGAAAAC-3′ |
| | (SEQ ID NO: 16) |
| | Reverse: 5′-AAACGTTTTCTCAGGCGCCGGCCC-3′ |
| | (SEQ ID NO: 17) |
| sgRNA VEGFA enhancer | Forward: 5′-CACCGCGCCTGAGTCAGAGAAGCC-3′ |
| | (SEQ ID NO: 18) |
| | Reverse: 5′-AAACGGCTTCTCTGACTCAGGCGC-3′ |
| | (SEQ ID NO: 19) |
| sgRAN3 HBB promoter | Forward: 5′-CACCGAATATTTGGAATCACAGCT-3′ |
| | (SEQ ID NO: 20) |
| | Reverse: 5′-AAACAGCTGTGATTCCAAATATTTC-3′ 3′ |
| | (SEQ ID NO: 21) |
| sgRNA4 HBB promoter | Forward: 5′-CACCGATTTGTGTAATAAGAAAAT-3′ |
| | (SEQ ID NO: 22) |
| | Reverse: 5′-AAACATTTTCTTATTACACAAATC-3′ 3′ |
| | (SEQ ID NO: 23) |
| sgRNA5 HBB promoter | Forward: 5′-CACCGTACGTAAATACACTTGCAA-3′ 3′ |
| | (SEQ ID NO: 24) |
| | Reverse: 5′-AAACTTGCAAGTGTATTTACGTAC-3′ 3′ |
| | (SEQ ID NO: 25) |
| sgRNA7 KCNE4 | Forward: 5′-CACCGGACTTCTTCTCCCGCCTCT-3′ |
| | (SEQ ID NO: 26) |
| | Reverse: 5′-AAACAGAGGCGGGAGAAGAAGTCC-3′ |
| | (SEQ ID NO: 27) |
| sgRNA8 KCNE4 | Forward: 5′-CACCGGGGCACCTGCACCGACCTC-3′ |
| | (SEQ ID NO: 28) |
| | Reverse: 5′-AAACGAGGTCGGTGCAGGTGCCCC-3′ |
| | (SEQ ID NO: 29) |
| sgRNA VEGFA promoter | Forward: 5′-CACCGGCTAGCACCAGCGCTCTGT-3′ 3′ |
| | (SEQ ID NO: 30) |
| | Reverse: 5′-AAACACAGAGCGCTGGTGCTAGCC-3′ |
| | (SEQ ID NO: 31) |

Cell Transfection:

K562 cells were maintained in RPMI 1640 supplemented with 10% FBS, 2 mM L-glutamin, 1 mM Sodium pyruvate and 1% penicillin-streptomycin. The cells were transfected using an Amaxa nucleofection device (Nucleofector™ 2 b). Two solutions were prepared for the transfection: solution 1 composed of 3.6M ATP-disodium Salt hydrate (Sigma, Cat# A2383), 0.6M MgCL2.6H₂0 (Sigma, Cat# M0250), 10 mL sterilized H₂O; solution 2 composed of 0.25M KH₂PO₄ (Sigma, Cat#7778-77-0), 0.033M NaHCO₃(Merck Millipore, Cat# L1703-BC), 5 mM Glucose (Sigma, Cat#50-99-7), H₂0 to reach 500 mL, NaOH (BioLab, Cat#1310-73-2) to reach pH 7.4. 80 μl. Solution 1 was mixed with 4 mL of solution 2.

0.5×10⁶ cells were seeded one day prior to transfection in each plate. On the day of tranfection, 1×10⁶ cells were centrifuged at 200 rcf for 5 minutes. The pellet was suspended with 100 μl of soultion 1 and 2 mix and with the plasmids, and transferred into 0.2 cm cuvettes (Mirus Bio, Cat# MC-MIR-50121). The cuvette was inserted to the Nucleofector and the T-016 program was chosen for the electroporation. After the program finished, the cells were seeded into plates with fresh medium. After 24 h, 2 μg/mL puromycin (Sigma Cat#P7255-25MG) was added to the medium. Real-time PCR: Total RNA was isolated from the cells with the use of Tri reagent (Bio-lab Cat#186-05-008) or by Rneasy kit (Qiagen Cat#1706005). Reverse transcription was carried out with a Verso cDNA Synthesis Kit (Thermo scientific Cat# AB-1453/B). The resulting cDNA was used as a template for RT PCR, which was performed with the Mx3005P device running MxPro QPCR software (Stratagene). Maxima SYBR Green/ROX qPCR Master mix (Thermo scientific Cat# K0221) was used to perform PCR. In genome editing experiment in VEGFA enhancer, hypoxanthine guanine phosphoribosyl transferase (HPRT) was used as a housekeeping gene to compensate for between-sample differences in the amount of cDNA. In genome editing experiment in PU.1 enhancer and in epigenetics editing experiment in HBB promoter, the genes were normalized with Glyceraldehyde 3-phosphate dehydrogenase (GAPDH) gene. All samples were amplified in triplicate and the data was analyzed with the use of MxPro qPCR system software (Stratagene). The primers are set forth in Table 2.

TABLE 2
qPCR primers sequences used in the experiments

| | |
|---|---|
| PU.1 | Forward: 5′-CGAGTATTACCCCTATCTCAGC-3′ |
| | (SEQ ID NO: 34) |
| | Reverse: 5′-CTGGTGGCCAAGACTGGG-3′ |
| | (SEQ ID NO: 35) |
| GAPDH | Forward: 5′-GCTCTCTGCTCCTCCTGTTC-3′ |
| | (SEQ ID NO: 36) |
| | Reverse: 5′-CGTTGACTCCGACCTTCAC-3′ |
| | (SEQ ID NO: 37) |
| HBB | Forward: 5′-CAAGGGCACCTTTGCCACAC-3′ |
| | (SEQ ID NO: 38) |
| | Reverse: 5′-TTTGCCAAAGTGATGGGCCA-3′ |
| | (SEQ ID NO: 39) |
| VEGFA | Forward: 5′-CTACCTCCACCATGCCAAGT-3′ |
| | (SEQ ID NO: 40) |
| | Reverse: 5′-GCAGTAGCTGCGCTGATAGA-3′ |
| | (SEQ ID NO: 41) |
| HPRT | Forward: 5′-TGACACTGGCAAAACAATGCA-3′ |
| | (SEQ ID NO: 42) |
| | Reverse: 5′-GGTCCTTTTCACCAGCAAGCT-3′ |
| | (SEQ ID NO: 43) |

DNA Extraction and Sequencing:

In the genome editing experiments, GFP positive cells were isolated as single cells by FACS. Genomic DNA was extracted (DNeasy Blood & Tissue Kit, Qiagen Cat#69504) from each clone, according to the manufacturer's protocol. The target region was amplified by PCR (primers are indicated in Table 3) and cloned into PGEM-T vector (Promega Corporation, Madison, Wis.). Following transformation of the vectors into TOP-10 (Life Technologies, Cat#440301) bacteria according to the manufacturer, the plasmids were purified using Nucleospin plasmid Easypure (Macherery-Nagel Cat# MAN-740727.250) and sequenced with T7 primer or SP6 primer.

TABLE 3
primers sequences for amplifying
the mutations regions

| | |
|---|---|
| PU.1 enhancer | Forward: 5′-CTTGGGTCTGGGGTCTGG-3′ |
| | (SEQ ID NO: 44) |
| | Reverse: 5′-CTGTGGTAATGGGCTGTTGG-3′ |
| | (SEQ ID NO: 45) |
| VEGFA enhancer | Forward: 5′-CCATCACTGCTCCACAATCA-3′ |
| | (SEQ ID NO: 46) |
| | Reverse: 5′-ACTCCGAGTGGCTCCTAGTG-3′ |
| | (SEQ ID NO: 47) |

High-Throughput Bisulfite Sequencing:

Genomic DNA was extracted (DNeasy) and bisulfite treated by using EZ DNA Methylation-Gold (Zymo research) according to the manufacturer's instructions. All samples underwent bisulfite conversion with an efficiency of at least 95% as determined by conversion of unmethylated, non-CpG cytosines. Genomic target sites were amplified by PCR using bisulfite-converted gDNA as a template with the primers in Table 4.

TABLE 4
primers sequences for amplifying the
target regions for sequencing.

| | |
|---|---|
| KCNE4 | forward: 5′-TCGTCGGCAGCGTCAGATGTGTATAAGAGAC |
| | AGGGAATTATGTTGGGTTATATGAAATTTAA-3′ |
| | (SEQ ID NO: 48) |
| | reverse: 5′-GTCTCGTGGGCTCGGAGATGTGTATAAGAGA |
| | CAGTCTACCCCCTCCTCCTAAATAATAA-3′ |
| | (SEQ ID NO: 49) |
| | forward: 5′-TCGTCGGCAGCGTCAGATGTGTATAAGAGAC |
| | AGTTTTTTTATGGAATAGAGGGTGTAG-3′ |
| | (SEQ ID NO: 50) |
| | reverse: 5′-GTCTCGTGGGCTCGGAGATGTGTATAAGAGA |
| | CAGACTTCTACATTCTAATTATCATATCCTTCT-3′ |
| | (SEQ ID NO: 51) |
| HBB | forward: 5′-GTCTCGTGGGCTCGGAGATGTGTATAAGAGA |
| | CAGGGATTTTAAATTTTTAGTTTTTTTT-3′ |
| | (SEQ ID NO: 52) |
| | reverse: 5′-TCGTCGGCAGCGTCAGATGTGTATAAGAGAC |
| | AGACTTTTAATACATCAACTTCTTATTTATAT-3′ |
| | (SEQ ID NO: 53) |
| VEGFA | forward: 5′-TCGTCGGCAGCGTCAGATGTGTATAAGAGAC |
| | AGGGTGTGAGTGGAATAATTTAAGTTTG-3′ |
| | (SEQ ID NO: 54) |
| | reverse: 5′-GTCTCGTGGGCTCGGAGATGTGTATAAGAGA |
| | CAGCATCCACCCTCTTTATAACCATTATAA-3′ |
| | (SEQ ID NO: 55) |
| PU.1 | forward: 5′-TCGTCGGCAGCGTCAGATGTGTATAAGAGAC |
| | AGGGGTTGTAGTTGTTTTTGTTTTTATAT-3′ |
| | (SEQ ID NO: 56) |
| | reverse: 5′-GTCTCGTGGGCTCGGAGATGTGTATAAGAGA |
| | CAGACTAAACATCCCCCTAAAACCTAAC-3′ |
| | (SEQ ID NO: 57) |

A second PCR was performed in order to add barcode sequences to each sample. Pooled amplicons were sequenced using an Illumina MiSeq with 150 bp single end-reads. For each experimental sample assayed, between 9619 to 279579 reads were analyzed. All samples underwent bisulfite conversion with an efficiency of at least 95% as judged by conversion of unmethylated, non-CpG cytosines.

Pyrosequencing in Targeting Dcas9-DNMT:

DNA was extracted and treated with bisulfite as mentioned above, CpG island in VEGFA was amplified by PCR by using the following primers: Forward: 5′-AAGAGGAAAGAGGTAGTAAGAGTT 3′ (SEQ ID NO: 58), Reverse: 5′-biotin-AATCACTCACTTTACCCCTATC 3′ (SEQ ID NO: 59). The PCR products were purified, quantified, and sequenced on a PyroMark Q24 bench-top device (Qiagen, Venlo, Limburg, the Netherlands) from the internal primer: 5′-AAGAGGTAGTAAGAGTTTT-3′ (SEQ ID NO: 60).

Results

To evaluate the efficiency of dcas9:TET demethylation, the present inventors initially target a methylated CpG island in K562 which is not targeted by transcription factors. This allows for the examination of the extent of the effect in nearby sites in accessible region without steric interference. Appropriate sgRNAs (Table 1 methods) were cloned into separate vectors under the U6 promoter.

Human K562 cells were transfected with 3 μg of plasmid encoding dcas9:TET or dcas9:TET inactive, 0.6 m from each one of the plasmids encoding the sgRNA sequence and 0.4 μg of GFP expressing plasmid. After 7 days, genomic DNA was isolated from the cells and methylation levels were determined by bisulfite treatment followed by high-throughput next-generation sequencing. The most significant demethylation effect (44-65%) was observed in 3 CpG sites at a distance of 18-50 bases downstream from gRNA8 PAM sequence (on strand -). The methylation level of 6 adjacent CpG sites was also reduced. However, the methylation in all examined CpG sites did not change by targeting the dcas9:TET bearing the inactivating mutations. Therefore, it may be concluded that the observed targeted methylation effect was not due to a steric effect (FIGS. 4A-B). The present inventors further validated that targeting dcas9:TET induced similar levels of demethylation on both DNA strands as expected.

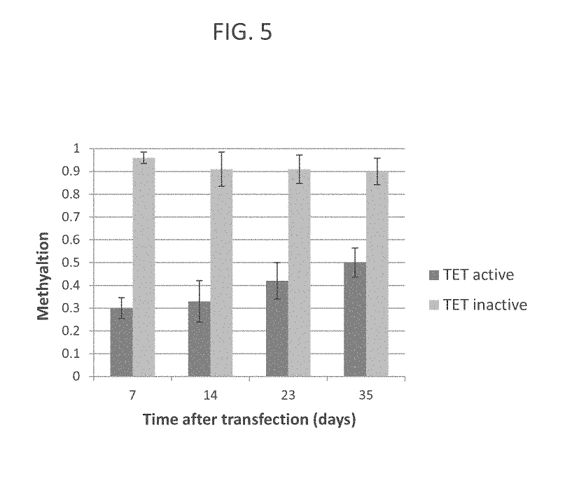

Next, the present inventors evaluated the time course of targeted demethylation effect by measuring the methylation levels in KCNE4 CGI at the following time points: 7, 14, 23 and 35 days following transfection in K562 cells. After 7 days, the methylation gradually elevated in all CpG sites examined, however the methylation levels did not return completely to the control methylation levels (FIG. 5). Similar trends were observed in other CpG sites in this region. Remethylation may be attributed to the fact that K562 cells have higher expression of de novo DNA methyltransferase (DNMT3A, DNMT3B) than the levels in normal hematopoiesis.

The present inventors next sought to determine whether targeted demethylation in key specific sites within a promoter may induce increase in gene expression. For this purpose, they chose to target the human beta globin (HBB) promoter in k562 cells, which has 4 CpG sites (FIG. 6A). CpG sites in HBB promoter are differentially methylated in erythroid cells isolated from fetal liver and adult bone marrow. Moreover, key transcription factor binding sites which are known to regulate globin gene GATA-1 and EKLF, are adjacent to these CpG sites.

The cells were transfected with 3 μg of plasmid encoding dcas9-TET or dcas9-TET inactive, 0.45 μg of sgRNA3 plasmid, 0.51 μg of sgRNA4 plasmid, 0.53 μg of sgRNA5 plasmid and 0.4 μg of GFP expressing plasmid. Five days following transfection, the DNA was purified and bisulfite treated and the methylation was evaluated by high throughput sequencing.

The methylation of CpG site at position −307 relative to HBB TSS (coordinate 5,248,607 in chromosome 11, FIG. 6B) was reduced significantly by 47% on average. This demethylation effect was specific since the methylation at the adjacent CpG site −266 upstream to HBB TSS (coordinate 5,248,566 in chromosome 11) did not change upon dcas9:TET targeting probably due to inaccessibility (FIG. 6B). Moreover, the methylation level at the experiments with targeting dcas9:TET inactive did not change at this CpG site.

Strikingly, the demethylation effect in the single CpG site was sufficient to induce change in HBB gene expression. HBB gene expression increased by 2.66 fold following the demethylation in the specific CpG site in FMB promoter compared to cells with targeted dcas9:TET inactive, 6 days after transfection (FIG. 7).

While the effects of mutations and methylation change on gene regulation have been well studied in gene promoters, these effects are unclear in distal regulatory elements. Thus, the present inventors chose to examine these effects on the well-established PU.1 enhancer and on the VEGFA enhancer.

PU.1 (SPI1) is an important hematopoietic transcription factor, and abnormal expression of SPI1 can lead to leukemia. The present inventors aimed to introduce mutations within the PU.1 enhancer in leukemia K562 cells since this region displays regulatory chromatin marks including DNaseI hypersensitivity, H3K4me1 and H3K27ac in these cells. Moreover, this region is abundant with transcription factor binding in K562 cells based on ENCODE CHIP-seq (The ENCODE Project Consortium. An integrated encyclopedia of DNA elements in the human genome. Nature 489, 57-74 (2012).

To design a CRISPR/Cas9 targeting PU.1 enhancer, a 19-bp nucleotide sequence adjacent to PU.1 binding core motif was chosen as the target site. It was hypothesized that this specific site plays a key role in PU.1 expression since it was shown that introducing mutations in the PU.1 core motif in this conserved enhancer in mice decreased the activity of a reporter gene by 100 fold (Okuno, Y. et al. Mol. Cell. Biol. 25, 2832-45 (2005)). K562 cells were transfected with 3.6 μg of cas9 and sgRNA plasmid and 0.4 μg GFP expressing (methods). 1 day after transfection, 60% of the cells were alive and transfection efficacy was high. Selection of the transfected cells was performed by using 4 μg/ml Puromycin for 4-5 days. The error-prone non-homologous end-joining repairing mechanism following CRISPR/Cas9 generates a heterogeneous population of genetic mutants. Thus, in order to evaluate the effect of specific mutations on SPI expression, GFP positive cells were isolated by FACS and single-cell clones were grown. Out of about 31 obtained clones, the two with the most significant effect on PU.1 expression were selected for downstream analysis (referred to herein as clone 30 and clone 31).

To verify the mutated sequences at the target sites, the targeted region was amplified by PCR using primers designed to amplify about 230 bp surrounding the target site.

Next, single allele sequencing analysis was performed due to the fact that K562 cells are known as near triploid and chromosome 11 has 2 or 3 homologues (there is cell-to-cell variation in the number of structurally normal chromosomes). For this analysis, the PCR products were cloned to a commercial plasmid and transformed to competent bacteria. Then, the plasmids were purified from different colonies and sequenced. This method allows for single allele sequencing since each bacteria can receive only one plasmid. The analysis revealed that the mutations in clone 30 were deletion of one to two bases in each allele in the target site whereas in clone 31 deletions of 5 or 10 bases in each allele were found.

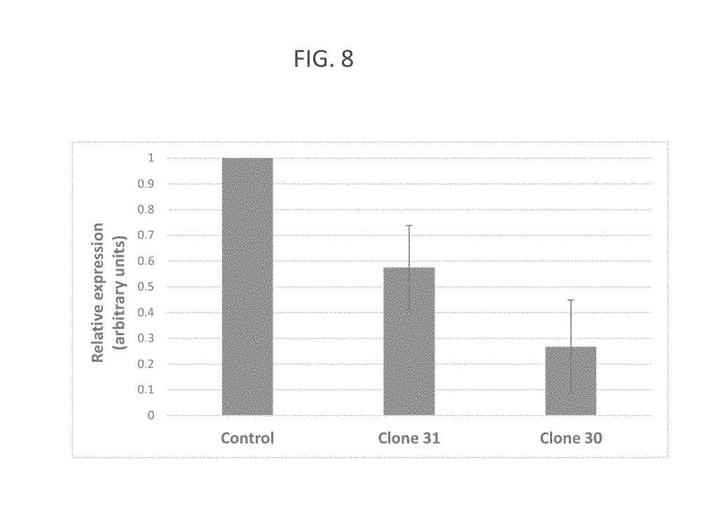

Strikingly, the small deletion in PU.1 enhancer significantly reduced PU.1 expression in the two clones. PU.1 expression was decreased by 1.7 fold in clone 31 and by 3.73 fold in clone 30 (FIG. 8). These results imply that the mutations affected critical specific key regulatory site within the PU.1 enhancer, which probably affected the binding of the transcription factors that regulate PU.1 expression.

The present inventors next investigated whether they could also identify key regulatory sites within the VEGFA enhancer. The cis-regulatory element of VEGFA gene, located 157 kb downstream from the promoter was shown to display regulatory chromatin marks including DNaseI hypersensitivity, H3K4me1 and H3K27ac in K562 cells. Multiple transcription factors are bound to this element based on ENCODE CHIP-seq data The ENCODE Project Consortium. An integrated encyclopedia of DNA elements in the human genome. Nature 489, 57-74 (2012). Moreover, there is negative correlation between the methylation of CpG site (chr6:43,894,639) in the regulatory element and VEGFA expression levels in ES, normal T and B cells, T cell leukemia (Jurkat) and erythroleukemia (K562) cells (Aran, D. et al. PLoS Genet. 12, (2016)).

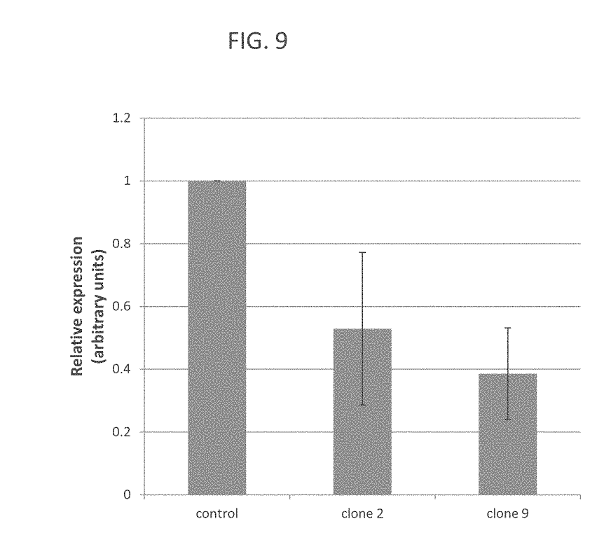

To explore whether the site near the correlative CpG site participates in VEGFA expression, a 19-bp nucleotide sequence sgRNA was designed which targeted the CpG site. Single allele sequencing analysis was performed as described previously, since K562 cells has 3 alleles of chromosome 6. The sgRNA efficiently induced Cas9-mediated indels in multiple clones of k562 cells. The mutations in the clones induced different effects on VEGFA expression, and two clones (referred to as clone 2 and clone 9) with the most significant downregulation effect on VEGFA expression were selected for downstream assays. The insertion of a single nucleotide of Adenine in the target site resulted in decrease of VEGFA expression by 1.88 fold and by 2.63 fold in clone 2 and 9 respectively as compared to mock-treated cells (FIG. 9).

Taken together, the results in the targeted mutation experiments in PU.1 enhancer and in VEGFA enhancer imply there are key sites within the regulatory element with a dominant effect on gene regulation.

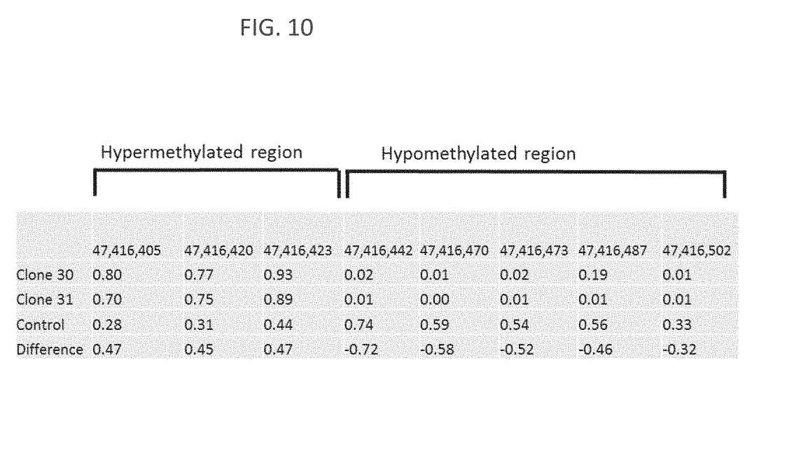

The present inventors next investigated whether the change in the DNA sequence and in gene expression was coupled with a change in the methylation of the CRISPR/Cas9 targeted region. They evaluated the methylation levels in 8 CpG sites in the sgRNA region by bisulfite followed by next-generation sequencing in the two clones with the down-regulation in PU.1 expression. Three CpG sites before the PAM sequence of the sgRNA were hypermethylated in the two clones by 47-45% compare to control cells without transfection. Whereas, five CpG sites downstream to the PAM sequence of the sgRNA were hypomethylated significantly by 32-72% compare to control cells (FIG. 10). These two regions may represent different regulatory regions.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1-21. (canceled)

22. A kit comprising:
a polynucleotide encoding a fusion protein which comprises a catalytically inactive CRISPR associated 9 (dCas9) protein linked to a TET protein; and
at least one guide RNA which is directed to an enhancer of a predetermined target gene.

23. (canceled)

24. A method of modifying DNA methylation of a target gene in a cell, the method comprising expressing a polynucleotide encoding a fusion protein which comprises a catalytically inactive CRISPR associated 9 (dCas9) protein linked to a TET protein in the cell, and one or more guide RNA directed to an enhancer of the target gene.

25. The method of claim 24, wherein the cell is a stem cell.

26. The method of claim 25, wherein said stem cell is a mesenchymal stem cell, an embryonic stem cell or an induced pluripotent stem cell.

27. The method of claim 24, wherein the cell is a cancer cell.

28. The method of claim 24, wherein said TET protein is TET1.

29. The method of claim 28, wherein said TET1 is human TET1.

30. The method of claim 24, wherein said TET protein comprises the catalytic domain of the TET protein.

31. The method of claim 30, wherein said catalytic domain of the TET protein comprises a sequence as least 90% identical to the sequence as set forth in SEQ ID NO: 1.

32. The method of claim 30, wherein said catalytic domain of the TET protein comprises a sequence 100% identical to the sequence as set forth in SEQ ID NO: 1.

33. The method of claim 30, wherein said catalytic domain is linked to said dCas9 via a peptide linker.

34. The method of claim 33, wherein said peptide linker comprises the sequence as set forth in SEQ ID NO: 3 (Gly, Gly, Gly, Gly, Ser).

35. The method of claim 24, wherein the catalytically inactive Cas9 protein comprises mutations at a site selected from the group consisting of D10, E762, H983, D986, H840 and N863.

36. The method of claim 35, wherein the mutations are: (i) D10A or D10N, and (ii) H840A, H840N, or H840Y.

37. The method of claim 24, wherein said dCAS9 comprises the sequence as set forth in SEQ ID NO: 2.

38. The method of claim 24, wherein said TET protein is linked to the C terminus of said dCas9.

39. The method of claim 24, wherein said TET protein is linked to the N terminus of said dCas9.

40. The method of claim 24, wherein said fusion protein comprises an amino acid sequence as set forth in SEQ ID NO: 4.

41. The method of claim 24 comprising a nucleic acid sequence as set forth in SEQ ID NO: 15.

42. The kit of claim 22, wherein said guide RNA is encoded from a nucleic acid construct.


SEQUENCE LISTING (61 sequences)

SEQ ID NO: 1 - protein, 2136 aa, Homo sapiens
MSRSRHARPSRLVRKEDVNKKKKNSQLRKTTKGANKNVASVKTLSPGKLKQLIQERDVKKKTEPKPPVPVRSLLTRAGAARMNLDRTEVLFQNPESLTCNGFTMALRSTSLSRRLSQPPLVVAKSKKVPLSKGLEKQHDCDYKILPALGVKHSENDSVPMQDTQVLPDIETLIGVQNPSLLKGKSQETTQFWSQRVEDSKINIPTHSGPAAEILPGPLEGTRCGEGLFSEETLNDTSGSPKMFAQDTVCAPFPQRATPKVTSQGNPSIQLEELGSRVESLKLSDSYLDPIKSEHDCYPTSSLNKVIPDLNLRNCLALGGSTSPTSVIKFLLAGSKQATLGAKPDHQEAFEATANQQEVSDTTSFLGQAFGAIPHQWELPGADPVHGEALGETPDLPEIPGAIPVQGEVFGTILDQQETLGMSGSVVPDLPVFLPVPPNPIATFNAPSKWPEPQSTVSYGLAVQGAIQILPLGSGHTPQSSSNSEKNSLPPVMAISNVENEKQVHISFLPANTQGFPLAPERGLFHASLGIAQLSQAGPSKSDRGSSQVSVTSTVHVVNTTVVTMPVPMVSTSSSSYTTLLPTLEKKKRKRCGVCEPCQQKTNCGECTYCKNRKNSHQICKKRKCEELKKKPSVVVPLEVIKENKRPQREKKPKVLKADFDNKPVNGPKSESMDYSRCGHGEEQKLELNPHTVENVTKNEDSMTGIEVEKWTQNKKSQLTDHVKGDFSANVPEAEKSKNSEVDKKRTKSPKLFVQTVRNGIKHVHCLPAETNVSFKKFNIEEFGKTLENNSYKFLKDTANHKNAMSSVATDMSCDHLKGRSNVLVFQQPGFNCSSIPHSSHSIINHHASIHNEGDQPKTPENIPSKEPKDGSPVQPSLLSLMKDRRLTLEQVVAIEALTQLSEAPSENSSPSKSEKDEESEQRTASLLNSCKAILYTVRKDLQDPNLQGEPPKLNHCPSLEKQSSCNTVVFNGQTTTLSNSHINSATNQASTKSHEYSKVTNSLSLFIPKSNSSKIDTNKSIAQGIITLDNCSNDLHQLPPRNNEVEYCNQLLDSSKKLDSDDLSCQDATHTQIEEDVATQLTQLASIIKINYIKPEDKKVESTPTSLVTCNVQQKYNQEKGTIQQKPPSSVHNNHGSSLTKQKNPTQKKTKSTPSRDRRKKKPTVVSYQENDRQKWEKLSYMYGTICDIWIASKFQNFGQFCPHDFPTVFGKISSSTKIWKPLAQTRSIMQPKTVFPPLTQIKLQRYPESAEEKVKVEPLDSLSLFHLKTESNGKAFTDKAYNSQVQLTVNANQKAHPLTQPSSPPNQCANVMAGDDQIRFQQVVKEQLMHQRLPTLPGISHETPLPESALTLRNVNVVCSGGITVVSTKSEEEVCSSSFGTSEFSTVDSAQKNFNDYAMNFFTNPTKNLVSITKDSELPTCSCLDRVIQKDKGPYYTHLGAGPSVAAVREIMENRYGQKGNAIRIEIVVYTGKEGKSSHGCPIAKWVLRRSSDEEKVLCLVRQRTGHHCPTAVMVVLIMVWDGIPLPMADRLYTELTENLKSYNGHPTDRRCTLNENRTCTCQGIDPETCGASFSFGCSWSMYFNGCKFGRSPSPRRFRIDPSSPLHEKNLEDNLQSLATRLAPIYKQYAPVAYQNQVEYENVARECRLGSKEGRPFSGVTACLDFCAHPHRDIHNMNNGSTVVCTLTREDNRSLGVIPQDEQLHVLPLYKLSDTDEFGSKEGMEAKIKSGAIEVLAPRRKKRTCFTQPVPRSGKKRAAMMTEVLAHKIRAVEKKPIPRIKRKNNSTTTNNSKPSSLPTLGSNTETVQPEVKSETEPHFILKSSDNTKTYSLMPSAPHPVKEASPGFSWSPKTASATPAPLKNDATASCGFSERSSTPHCTMPSGRLSGANAAAADGPGISQLGEVAPLPTLSAPVMEPLINSEPSTGVTEPLTPHQPNHQPSFLTSPQDLASSPMEEDEQHSEADEPPSDEPLSDDPLSPAEEKLPHIDEYWSDSEHIFLDANIGGVAIAPAHGSVLIECARRELHATTPVEHPNRNHPTRLSLVFYQHKNLNKPQHGFELNKIKFEAKEAKNKKMKASEQKDQAANEGPEQSSEVNELNQIPSHKALTLTHDNVVTVSPYALTHVAGPYNHWV

SEQ ID NO: 2 - protein, 1367 aa, Artificial sequence
  sequence of the catalytically inactive S.      pyogenes Cas9
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 3 - protein, 5 aa, Artificial sequence
  linker peptide sequence
GGGGS

SEQ ID NO: 4 - protein, 2125 aa, Artificial sequence
  fusion protein (HA epitope dCAS9, NLS, LINKER,      TET1 catalytic domain)
MYPYDVPDYASPKKKRKVEASDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSPKKKRKVEASGPAGGGGSLPTCSCLDRVIQKDKGPYYTHLGAGPSVAAVREIMENRYGQKGNAIRIEIVVYTGKEGKSSHGCPIAKWVLRRSSDEEKVLCLVRQRTGHHCPTAVMVVLIMVWDGIPLPMADRLYTELTENLKSYNGHPTDRRCTLNENRTCTCQGIDPETCGASFSFGCSWSMYFNGCKFGRSPSPRRFRIDPSSPLHEKNLEDNLQSLATRLAPIYKQYAPVAYQNQVEYENVARECRLGSKEGRPFSGVTACLDFCAHPHRDIHNMNNGSTVVCTLTREDNRSLGVIPQDEQLHVLPLYKLSDTDEFGSKEGMEAKIKSGAIEVLAPRRKKRTCFTQPVPRSGKKRAAMMTEVLAHKIRAVEKKPIPRIKRKNNSTTTNNSKPSSLPTLGSNTETVQPEVKSETEPHFILKSSDNTKTYSLMPSAPHPVKEASPGFSWSPKTASATPAPLKNDATASCGFSERSSTPHCTMPSGRLSGANAAAADGPGISQLGEVAPLPTLSAPVMEPLINSEPSTGVTEPLTPHQPNHQPSFLTSPQDLASSPMEEDEQHSEADEPPSDEPLSDDPLSPAEEKLPHIDEYWSDSEHIFLDANIGGVAIAPAHGSVLIECARRELHATTPVEHPNRNHPTRLSLVFYQHKNLNKPQHGFELNKIKFEAKEAKNKKMKASEQKDQAANEGPEQSSEVNELNQIPSHKALTLTHDNVVTVSPYALTHVAGPYNHWV

SEQ ID NO: 5 - DNA, 4101 bp, Artificial sequence
  Single strand DNA oligonucleotide
gacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgccatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgac

SEQ ID NO: 6 - DNA, 9601 bp, Homo sapiens
agacactgctgctccggggggctgacctggcggggagtggccgcgcagtctgctccggcgccgctttgtgcgcgcagccgctggcccctctactcccgggtctgccccccgggacacccctctgcctcgcccaagtcatgcagccctacctgcctctccactgtggacctttgggaaccgactcctcacctcgggggctcgggccttgactgtgctgggagccggtaggcgtcctccgcgacccgcccgcgcccctcgcgcccgccggggccccgggctccaaagttgtggggaccggcgcgagttggaaagtttgcccgagggctggtgcaggcttggagctgggggccgtgcgctgccctgggaatgtgacccggccagcgaccaaaaccttgtgtgactgagctgaagagcagtgcatccagattctcctcagaagtgagactttccaaaggaccaatgactctgtttcctgcgccctttcattttttcctactctgtagctatgtctcgatcccgccatgcaaggccttccagattagtcaggaaggaagatgtaaacaaaaaaaagaaaaacagccaactacgaaagacaaccaagggagccaacaaaaatgtggcatcagtcaagactttaagccctggaaaattaaagcaattaattcaagaaagagatgttaagaaaaaaacagaacctaaaccacccgtgccagtcagaagccttctgacaagagctggagcagcacgcatgaatttggataggactgaggttctttttcagaacccagagtccttaacctgcaatgggtttacaatggcgctacgaagcacctctcttagcaggcgactctcccaacccccactggtcgtagccaaatccaaaaaggttccactttctaagggtttagaaaagcaacatgattgtgattataagatactccctgctttgggagtaaagcactcagaaaatgattcggttccaatgcaagacacccaagtccttcctgatatagagactctaattggtgtacaaaatccctctttacttaaaggtaagagccaagagacaactcagttttggtcccaaagagttgaggattccaagatcaatatccctacccacagtggccctgcagctgagatccttcctgggccactggaagggacacgctgtggtgaaggactattctctgaagagacattgaatgataccagtggttccccaaaaatgtttgctcaggacacagtgtgtgctccttttccccaaagagcaacccccaaagttacctctcaaggaaaccccagcattcagttagaagagttgggttcacgagtagaatctcttaagttatctgattcttacctggatcccattaaaagtgaacatgattgctaccccacctccagtcttaataaggttatacctgacttgaaccttagaaactgcttggctcttggtgggtctacgtctcctacctctgtaataaaattcctcttggcaggctcaaaacaagcgacccttggtgctaaaccagatcatcaagaggccttcgaagctactgcaaatcaacaggaagtttctgataccacctctttcctaggacaggcctttggtgctatcccacatcaatgggaacttcctggtgctgacccagttcatggtgaggccctgggtgagaccccagatctaccagagattcctggtgctattccagtccaaggagaggtctttggtactattttagaccaacaagaaactcttggtatgagtgggagtgttgtcccagacttgcctgtcttccttcctgttcctccaaatccaattgctacctttaatgctccttccaaatggcctgagccccaaagcactgtctcatatggacttgcagtccagggtgctatacagattttgcctttgggctcaggacacactcctcaatcatcatcaaactcagagaaaaattcattacctccagtaatggctataagcaatgtagaaaatgagaagcaggttcatataagcttcctgccagctaacactcaggggttcccattagcccctgagagaggactcttccatgcttcactgggtatagcccaactctctcaggctggtcctagcaaatcagacagagggagctcccaggtcagtgtaaccagcacagttcatgttgtcaacaccacagtggtgactatgccagtgccaatggtcagtacctcctcttcttcctataccactttgctaccgactttggaaaagaagaaaagaaagcgatgtggggtctgtgaaccctgccagcagaagaccaactgtggtgaatgcacttactgcaagaacagaaagaacagccatcagatctgtaagaaaagaaaatgtgaggagctgaaaaagaaaccatctgttgttgtgcctctggaggttataaaggaaaacaagaggccccagagggaaaagaagcccaaagttttaaaggcagattttgacaacaaaccagtaaatggccccaagtcagaatccatggactacagtagatgtggtcatggggaagaacaaaaattggaattgaacccacatactgttgaaaatgtaactaaaaatgaagacagcatgacaggcatcgaggtggagaagtggacacaaaacaagaaatcacagttaactgatcacgtgaaaggagattttagtgctaatgtcccagaagctgaaaaatcgaaaaactctgaagttgacaagaaacgaaccaaatctccaaaattgtttgtacaaaccgtaagaaatggcattaaacatgtacactgtttaccagctgaaacaaatgtttcatttaaaaaattcaatattgaagaattcggcaagacattggaaaacaattcttataaattcctaaaagacactgcaaaccataaaaacgctatgagctctgttgctactgatatgagttgtgatcatctcaaggggagaagtaacgttttagtattccagcagcctggctttaactgcagttccattccacattcttcacactccatcataaatcatcatgctagtatacacaatgaaggtgatcaaccaaaaactcctgagaatataccaagtaaagaaccaaaagatggatctcccgttcaaccaagtctcttatcgttaatgaaagataggagattaacattggagcaagtggtagccatagaggccctgactcaactctcagaagccccatcagagaattcctccccatcaaagtcagagaaggatgaggaatcagagcagagaacagccagtttgcttaatagctgcaaagctatcctctacactgtaagaaaagacctccaagacccaaacttacagggagagccaccaaaacttaatcactgtccatctttggaaaaacaaagttcatgcaacacggtggttttcaatgggcaaactactaccctttccaactcacatatcaactcagctactaaccaagcatccacaaagtcacatgaatattcaaaagtcacaaattcattatctctttttataccaaaatcaaattcatccaagattgacaccaataaaagtattgctcaagggataattactcttgacaattgttccaatgatttgcatcagttgccaccaagaaataatgaagtggagtattgcaaccagttactggacagcagcaaaaaattggactcagatgatctatcatgtcaggatgcaacccatacccaaattgaggaagatgttgcaacacagttgacacaacttgcttcgataattaagatcaattatataaaaccagaggacaaaaaagttgaaagtacaccaacaagccttgtcacatgtaatgtacagcaaaaatacaatcaggagaagggcacaatacaacagaaaccaccttcaagtgtacacaataatcatggttcatcattaacaaaacaaaagaacccaacccagaaaaagacaaaatccaccccatcaagagatcggcggaaaaagaagcccacagttgtaagttatcaagaaaatgatcggcagaagtgggaaaagttgtcctatatgtatggcacaatatgcgacatttggatagcatcgaaatttcaaaattttgggcaattttgtccacatgattttcctactgtatttgggaaaatttcttcctcgaccaaaatatggaaaccactggctcaaacgaggtccattatgcaacccaaaacagtatttccaccactcactcagataaaattacagagatatcctgaatcagcagaggaaaaggtgaaggttgaaccattggattcactcagcttatttcatcttaaaacggaatccaacgggaaggcattcactgataaagcttataattctcaggtacagttaacggtgaatgccaatcagaaagcccatcctttgacccagccctcctctccacctaaccagtgtgctaacgtgatggcaggcgatgaccaaatacggtttcagcaggttgttaaggagcaactcatgcatcagagactgccaacattgcctggtatctctcatgaaacacccttaccggagtcagcactaactctcaggaatgtaaatgtagtgtgttcaggtggaattacagtggtttctaccaaaagtgaagaggaagtctgttcatccagttttggaacatcagaattttccacagtggacagtgcacagaaaaattttaatgattatgccatgaacttctttactaaccctacaaaaaacctagtgtctataactaaagattctgaactgcccacctgcagctgtcttgatcgagttatacaaaaagacaaaggcccatattatacacaccttggggcaggaccaagtgttgctgctgtcagggaaatcatggagaataggtatggtcaaaaaggaaacgcaataaggatagaaatagtagtgtacaccggtaaagaagggaaaagctctcatgggtgtccaattgctaagtgggttttaagaagaagcagtgatgaagaaaaagttctttgtttggtccggcagcgtacaggccaccactgtccaactgctgtgatggtggtgctcatcatggtgtgggatggcatccctcttccaatggccgaccggctatacacagagctcacagagaatctaaagtcatacaatgggcaccctaccgacagaagatgcaccctcaatgaaaatcgtacctgtacatgtcaaggaattgatccagagacttgtggagcttcattctcttttggctgttcatggagtatgtactttaatggctgtaagtttggtagaagcccaagccccagaagatttagaattgatccaagctctcccttacatgaaaaaaaccttgaagataacttacagagtttggctacacgattagctccaatttataagcagtatgctccagtagcttaccaaaatcaggtggaatatgaaaatgttgcccgagaatgtcggcttggcagcaaggaaggtcgtcccttctctggggtcactgcttgcctggacttctgtgctcatccccacagggacattcacaacatgaataatggaagcactgtggtttgtaccttaactcgagaagataaccgctctttgggtgttattcctcaagatgagcagctccatgtgctacctctttataagctttcagacacagatgagtttggctccaaggaaggaatggaagccaagatcaaatctggggccatcgaggtcctggcaccccgccgcaaaaaaagaacgtgtttcactcagcctgttccccgttctggaaagaagagggctgcgatgatgacagaggttcttgcacataagataagggcagtggaaaagaaacctattccccgaatcaagcggaagaataactcaacaacaacaaacaacagtaagccttcgtcactgccaaccttagggagtaacactgagaccgtgcaacctgaagtaaaaagtgaaaccgaaccccattttatcttaaaaagttcagacaacactaaaacttattcgctgatgccatccgctcctcacccagtgaaagaggcatctccaggcttctcctggtccccgaagactgcttcagccacaccagctccactgaagaatgacgcaacagcctcatgcgggttttcagaaagaagcagcactccccactgtacgatgccttcgggaagactcagtggtgccaatgcagctgctgctgatggccctggcatttcacagcttggcgaagtggctcctctccccaccctgtctgctcctgtgatggagcccctcattaattctgagccttccactggtgtgactgagccgctaacgcctcatcagccaaaccaccagccctccttcctcacctctcctcaagaccttgcctcttctccaatggaagaagatgagcagcattctgaagcagatgagcctccatcagacgaacccctatctgatgaccccctgtcacctgctgaggagaaattgccccacattgatgagtattggtcagacagtgagcacatctttttggatgcaaatattggtggggtggccatcgcacctgctcacggctcggttttgattgagtgtgcccggcgagagctgcacgctaccactcctgttgagcaccccaaccgtaatcatccaacccgcctctcccttgtcttttaccagcacaaaaacctaaataagccccaacatggttttgaactaaacaagattaagtttgaggctaaagaagctaagaataagaaaatgaaggcctcagagcaaaaagaccaggcagctaatgaaggtccagaacagtcctctgaagtaaatgaattgaaccaaattccttctcataaagcattaacattaacccatgacaatgttgtcaccgtgtccccttatgctctcacacacgttgcggggccctataaccattgggtctgaaggcttttctccccctcttaatgcctttgctagtgcagtgtattttttcaaggtgctgttaaaagaaagtcatgttgtcgtttactatcttcatctcacccatttcaagtctgaggtaaaaaaataataatgataacaaaacggggtgggtattcttaactgtgactatattttgacaattggtagaaggtgcacattttaagcaaaaataaaagttttatagttttaaatacataaagaaatgtttcagttaggcattaaccttgatagaatcactcagtttggtgctttaaattaagtctgtttactatgaaacaagagtcatttttagaggattttaacaggttcatgttctatgatgtaaaatcaagacacacagtgttaactctacacagcttctggtgcttaaccacatccacacagttaaaaataagctgaattattatttcatggtgccattgttccaacatcttccaatcattgctagaaaattggcatattcctttgaaataaacttatgaaatgttttctctcttaaaatatttctcctgtgtaaaataaatcattgttgttagtaatggttggaggctgttcataaattgtaaatatatattttaaaagcactttctatttttaaaagtaacttgaaataatatagtataagaatcctattgtctattgtttgtgcatatttgcatacaagagaaatcatttatccttgctgtgtagagttccatcttgttaactgcagtatgtattctaatcatgtatatggtttgtgttcttttactgtgtcctctcacattcaagtattagcaacttgcagtatataaaatagttagataatgagaagttgttaattatctctaaaattggaattaggaagcatatcaccaatactgattaacattctctttggaactaggtaagagtggtctcttcttattgaacaacctcaatttagtttcatcccacctttctcagtataatccatgagaggtgtttccaaaaggagatgagggaacaggataggtttcagaagagtcaaatgcttctaatgtctcaaggtgataaaatacaaaaactaagtagacagatatttgtactgaagtctgatacagaattagaaaaaaaaaattcttgttgaaatattttgaaaacaaattccctactatcatcacatgcctccccaaccccaagtcaaaaacaagaggaatggtactacaaacatggctttgtccattaagagctaattcatttgtttatcttagcatactagatttgggaaaatgataactcatcttttctgataattgcctatgttctaggtaacaggaaaacaggcattaagtttattttagtcttcccattttcttcctattactttattgactcattttattgcaaaacaaaaaggattacccaaacaacatgtttcgaacaaggagaattttcaatgaaatacttgattctgttaaaatgcagaggtgctataacattcaaagtgtcagattccttgggagtatggaaaacctaatggtgcttctcccttggaaatgccataggaagcccacaaccgctaacacttacaattttggtgcaaaagcaaacagttccagcaggctctctaaagaaaaactcattgtaacttattaaaataatatctggtgcaaagtatctgttttgagcttttgactaatccaagtaaaggaatatgaagggattgtaaaaaacaaaatgtccattgatagaccatcgtgtacaagtagatttctgcttgttgaatatgtaaaatagggtaattcattgacttgttttagtattttgtgtgccttagatttccgttttaagacatgtatatttttgtgagcctaaggtttcttatatacatataagtatataaataagtgattgtttattgcttcagctgcttcaacaagatatttactagtattagactatcaggaatacacccttgcgagattatgttttagattttaggccttagctcccactagaaattatttcttcaccagatttaatggataaagttttatggctctttatgcatccactcatctactcattcttcgagtctacacttattgaatgcctgcaaaatctaagtatcacttttatttttctttggatcaccacctatgacatagtaaacttgaagaataaaaactaccctcagaaatatttttaaaagaagtagcaaattatcttcagtataatccatggtaatgtatgcagtaattcaaattgatctctctctcaataggtttcttaacaatctaaacttgaaacatcaatgttaatttttggaactattgggatttgtgacgcttgttgcagtttaccaaaacaagtatttgaaaatatatagtatcaactgaaatgtttccattccgttgttgtagttaacatcatgaatggacttcttaagctgattaccccactgtgggaaccaaattggattcctactttgttggactctctttcctgattttaacaatttaccatcccattctctgccctgtgattttttttaaaagcttattcaatgttctgcagcattgtgattgtatgctggctacactgcttttagaatgctctttctcatgaagcaaggaaataaatttgtttgaaatgacattttctctcaaaaaaaaaaaaaaaaaa

SEQ ID NO: 7 - protein, 718 aa, Homo sapiens
LPTCSCLDRVIQKDKGPYYTHLGAGPSVAAVREIMENRYGQKGNAIRIEIVVYTGKEGKSSHGCPIAKWVLRRSSDEEKVLCLVRQRTGHHCPTAVMVVLIMVWDGIPLPMADRLYTELTENLKSYNGHPTDRRCTLNENRTCTCQGIDPETCGASFSFGCSWSMYFNGCKFGRSPSPRRFRIDPSSPLHEKNLEDNLQSLATRLAPIYKQYAPVAYQNQVEYENVARECRLGSKEGRPFSGVTACLDFCAHPHRDIHNMNNGSTVVCTLTREDNRSLGVIPQDEQLHVLPLYKLSDTDEFGSKEGMEAKIKSGAIEVLAPRRKKRTCFTQPVPRSGKKRAAMMTEVLAHKIRAVEKKPIPRIKRKNNSTTTNNSKPSSLPTLGSNTETVQPEVKSETEPHFILKSSDNTKTYSLMPSAPHPVKEASPGFSWSPKTASATPAPLKNDATASCGFSERSSTPHCTMPSGRLSGANAAAADGPGISQLGEVAPLPTLSAPVMEPLINSEPSTGVTEPLTPHQPNHQPSFLTSPQDLASSPMEEDEQHSEADEPPSDEPLSDDPLSPAEEKLPHIDEYWSDSEHIFLDANIGGVAIAPAHGSVLIECARRELHATTPVEHPNRNHPTRLSLVFYQHKNLNKPQHGFELNKIKFEAKEAKNKKMKASEQKDQAANEGPEQSSEVNELNQIPSHKALTLTHDNVVTVSPYALTHVAGPYNHWV

SEQ ID NO: 8 - DNA, 2154 bp, Homo sapiens
ctgcccacctgcagctgtcttgatcgagttatacaaaaagacaaaggcccatattatacacaccttggggcaggaccaagtgttgctgctgtcagggaaatcatggagaataggtatggtcaaaaaggaaacgcaataaggatagaaatagtagtgtacaccggtaaagaagggaaaagctctcatgggtgtccaattgctaagtgggttttaagaagaagcagtgatgaagaaaaagttctttgtttggtccggcagcgtacaggccaccactgtccaactgctgtgatggtggtgctcatcatggtgtgggatggcatccctcttccaatggccgaccggctatacacagagctcacagagaatctaaagtcatacaatgggcaccctaccgacagaagatgcaccctcaatgaaaatcgtacctgtacatgtcaaggaattgatccagagacttgtggagcttcattctcttttggctgttcatggagtatgtactttaatggctgtaagtttggtagaagcccaagccccagaagatttagaattgatccaagctctcccttacatgaaaaaaaccttgaagataacttacagagtttggctacacgattagctccaatttataagcagtatgctccagtagcttaccaaaatcaggtggaatatgaaaatgttgcccgagaatgtcggcttggcagcaaggaaggtcgtcccttctctggggtcactgcttgcctggacttctgtgctcatccccacagggacattcacaacatgaataatggaagcactgtggtttgtaccttaactcgagaagataaccgctctttgggtgttattcctcaagatgagcagctccatgtgctacctctttataagctttcagacacagatgagtttggctccaaggaaggaatggaagccaagatcaaatctggggccatcgaggtcctggcaccccgccgcaaaaaaagaacgtgtttcactcagcctgttccccgttctggaaagaagagggctgcgatgatgacagaggttcttgcacataagataagggcagtggaaaagaaacctattccccgaatcaagcggaagaataactcaacaacaacaaacaacagtaagccttcgtcactgccaaccttagggagtaacactgagaccgtgcaacctgaagtaaaaagtgaaaccgaaccccattttatcttaaaaagttcagacaacactaaaacttattcgctgatgccatccgctcctcacccagtgaaagaggcatctccaggcttctcctggtccccgaagactgcttcagccacaccagctccactgaagaatgacgcaacagcctcatgcgggttttcagaaagaagcagcactccccactgtacgatgccttcgggaagactcagtggtgccaatgcagctgctgctgatggccctggcatttcacagcttggcgaagtggctcctctccccaccctgtctgctcctgtgatggagcccctcattaattctgagccttccactggtgtgactgagccgctaacgcctcatcagccaaaccaccagccctccttcctcacctctcctcaagaccttgcctcttctccaatggaagaagatgagcagcattctgaagcagatgagcctccatcagacgaacccctatctgatgaccccctgtcacctgctgaggagaaattgccccacattgatgagtattggtcagacagtgagcacatctttttggatgcaaatattggtggggtggccatcgcacctgctcacggctcggttttgattgagtgtgcccggcgagagctgcacgctaccactcctgttgagcaccccaaccgtaatcatccaacccgcctctcccttgtcttttaccagcacaaaaacctaaataagccccaacatggttttgaactaaacaagattaagtttgaggctaaagaagctaagaataagaaaatgaaggcctcagagcaaaaagaccaggcagctaatgaaggtccagaacagtcctctgaagtaaatgaattgaaccaaattccttctcataaagcattaacattaacccatgacaatgttgtcaccgtgtccccttatgctctcacacacgttgcggggccctataaccattgggtc

SEQ ID NO: 9 - protein, 2002 aa, Homo sapiens
MEQDRTNHVEGNRLSPFLIPSPPICQTEPLATKLQNGSPLPERAHPEVNGDTKWHSFKSYYGIPCMKGSQNSRVSPDFTQESRGYSKCLQNGGIKRTVSEPSLSGLLQIKKLKQDQKANGERRNFGVSQERNPGESSQPNVSDLSDKKESVSSVAQENAVKDFTSFSTHNCSGPENPELQILNEQEGKSANYHDKNIVLLKNKAVLMPNGATVSASSVEHTHGELLEKTLSQYYPDCVSIAVQKTTSHINAINSQATNELSCEITHPSHTSGQINSAQTSNSELPPKPAAVVSEACDADDADNASKLAAMLNTCSFQKPEQLQQQKSVFEICPSPAENNIQGTTKLASGEEFCSGSSSNLQAPGGSSERYLKQNEMNGAYFKQSSVFTKDSFSATTTPPPPSQLLLSPPPPLPQVPQLPSEGKSTLNGGVLEEHHHYPNQSNTTLLREVKIEGKPEAPPSQSPNPSTHVCSPSPMLSERPQNNCVNRNDIQTAGTMTVPLCSEKTRPMSEHLKHNPPIFGSSGELQDNCQQLMRNKEQEILKGRDKEQTRDLVPPTQHYLKPGWIELKAPRFHQAESHLKRNEASLPSILQYQPNLSNQMTSKQYTGNSNMPGGLPRQAYTQKTTQLEHKSQMYQVEMNQGQSQGTVDQHLQFQKPSHQVHFSKTDHLPKAHVQSLCGTRFHFQQRADSQTEKLMSPVLKQHLNQQASETEPFSNSHLLQHKPHKQAAQTQPSQSSHLPQNQQQQQKLQIKNKEEILQTFPHPQSNNDQQREGSFFGQTKVEECFHGENQYSKSSEFETHNVQMGLEEVQNINRRNSPYSQTMKSSACKIQVSCSNNTHLVSENKEQTTHPELFAGNKTQNLHHMQYFPNNVIPKQDLLHRCFQEQEQKSQQASVLQGYKNRNQDMSGQQAAQLAQQRYLIHNHANVFPVPDQGGSHTQTPPQKDTQKHAALRWHLLQKQEQQQTQQPQTESCHSQMHRPIKVEPGCKPHACMHTAPPENKTWKKVTKQENPPASCDNVQQKSIIETMEQHLKQFHAKSLFDHKALTLKSQKQVKVEMSGPVTVLTRQTTAAELDSHTPALEQQTTSSEKTPTKRTAASVLNNFIESPSKLLDTPIKNLLDTPVKTQYDFPSCRCVEQIIEKDEGPFYTHLGAGPNVAAIREIMEERFGQKGKAIRIERVIYTGKEGKSSQGCPIAKWVVRRSSSEEKLLCLVRERAGHTCEAAVIVILILVWEGIPLSLADKLYSELTETLRKYGTLTNRRCALNEERTCACQGLDPETCGASFSFGCSWSMYYNGCKFARSKIPRKFKLLGDDPKEEEKLESHLQNLSTLMAPTYKKLAPDAYNNQIEYEHRAPECRLGLKEGRPFSGVTACLDFCAHAHRDLHNMQNGSTLVCTLTREDNREFGGKPEDEQLHVLPLYKVSDVDEFGSVEAQEEKKRSGAIQVLSSFRRKVRMLAEPVKTCRQRKLEAKKAAAEKLSSLENSSNKNEKEKSAPSRTKQTENASQAKQLAELLRLSGPVMQQSQQPQPLQKQPPQPQQQQRPQQQQPHHPQTESVNSYSASGSTNPYMRRPNPVSPYPNSSHTSDIYGSTSPMNFYSTSSQAAGSYLNSSNPMNPYPGLLNQNTQYPSYQCNGNLSVDNCSPYLGSYSPQSQPMDLYRYPSQDPLSKLSLPPIHTLYQPRFGNSQSFTSKYLGYGNQNMQGDGFSSCTIRPNVHHVGKLPPYPTHEMDGHFMGATSRLPPNLSNPNMDYKNGEHHSPSHIIHNYSAAPGMFNSSLHALHLQNKENDMLSHTANGLSKMLPALNHDRTACVQGGLHKLSDANGQEKQPLALVQGVASGAEDNDEVWSDSEQSFLDPDIGGVAVAPTHGSILIECAKRELHATTPLKNPNRNHPTRISLVFYQHKSMNEPKHGLALWEAKMAEKAREKEEECEKYGPDYVPQKSHGKKVKREPAEPHETSEPTYLRFIKSLAERTMSVTTDSTVTTSPYAFTRVTGPYNRYI

SEQ ID NO: 10 - DNA, 9796 bp, Homo sapiens
ggcagtggcagcggcgagagcttgggcggccgccgccgcctcctcgcgagcgccgcgcgcccgggtcccgctcgcatgcaagtcacgtccgccccctcggcgcggccgccccgagacgccggccccgctgagtgatgagaacagacgtcaaactgccttatgaatattgatgcggaggctaggctgctttcgtagagaagcagaaggaagcaagatggctgccctttaggatttgttagaaaggagacccgactgcaactgctggattgctgcaaggctgagggacgagaacgaggctggcaaacattcagcagcacaccctctcaagattgtttacttgcctttgctcctgttgagttacaacgcttggaagcaggagatgggctcagcagcagccaataggacatgatccaggaagagcagtaagggactgagctgctgaattcaactagagggcagccttgtggatggccccgaagcaagcctgatggaacaggatagaaccaaccatgttgagggcaacagactaagtccattcctgataccatcacctcccatttgccagacagaacctctggctacaaagctccagaatggaagcccactgcctgagagagctcatccagaagtaaatggagacaccaagtggcactctttcaaaagttattatggaataccctgtatgaagggaagccagaatagtcgtgtgagtcctgactttacacaagaaagtagagggtattccaagtgtttgcaaaatggaggaataaaacgcacagttagtgaaccttctctctctgggctccttcagatcaagaaattgaaacaagaccaaaaggctaatggagaaagacgtaacttcggggtaagccaagaaagaaatccaggtgaaagcagtcaaccaaatgtctccgatttgagtgataagaaagaatctgtgagttctgtagcccaagaaaatgcagttaaagatttcaccagtttttcaacacataactgcagtgggcctgaaaatccagagcttcagattctgaatgagcaggaggggaaaagtgctaattaccatgacaagaacattgtattacttaaaaacaaggcagtgctaatgcctaatggtgctacagtttctgcctcttccgtggaacacacacatggtgaactcctggaaaaaacactgtctcaatattatccagattgtgtttccattgcggtgcagaaaaccacatctcacataaatgccattaacagtcaggctactaatgagttgtcctgtgagatcactcacccatcgcatacctcagggcagatcaattccgcacagacctctaactctgagctgcctccaaagccagctgcagtggtgagtgaggcctgtgatgctgatgatgctgataatgccagtaaactagctgcaatgctaaatacctgttcctttcagaaaccagaacaactacaacaacaaaaatcagtttttgagatatgcccatctcctgcagaaaataacatccagggaaccacaaagctagcgtctggtgaagaattctgttcaggttccagcagcaatttgcaagctcctggtggcagctctgaacggtatttaaaacaaaatgaaatgaatggtgcttacttcaagcaaagctcagtgttcactaaggattccttttctgccactaccacaccaccaccaccatcacaattgcttctttctccccctcctcctcttccacaggttcctcagcttccttcagaaggaaaaagcactctgaatggtggagttttagaagaacaccaccactaccccaaccaaagtaacacaacacttttaagggaagtgaaaatagagggtaaacctgaggcaccaccttcccagagtcctaatccatctacacatgtatgcagcccttctccgatgctttctgaaaggcctcagaataattgtgtgaacaggaatgacatacagactgcagggacaatgactgttccattgtgttctgagaaaacaagaccaatgtcagaacacctcaagcataacccaccaatttttggtagcagtggagagctacaggacaactgccagcagttgatgagaaacaaagagcaagagattctgaagggtcgagacaaggagcaaacacgagatcttgtgcccccaacacagcactatctgaaaccaggatggattgaattgaaggcccctcgttttcaccaagcggaatcccatctaaaacgtaatgaggcatcactgccatcaattcttcagtatcaacccaatctctccaatcaaatgacctccaaacaatacactggaaattccaacatgcctggggggctcccaaggcaagcttacacccagaaaacaacacagctggagcacaagtcacaaatgtaccaagttgaaatgaatcaagggcagtcccaaggtacagtggaccaacatctccagttccaaaaaccctcacaccaggtgcacttctccaaaacagaccatttaccaaaagctcatgtgcagtcactgtgtggcactagatttcattttcaacaaagagcagattcccaaactgaaaaacttatgtccccagtgttgaaacagcacttgaatcaacaggcttcagagactgagccattttcaaactcacaccttttgcaacataagcctcataaacaggcagcacaaacacaaccatcccagagttcacatctccctcaaaaccagcaacagcagcaaaaattacaaataaagaataaagaggaaatactccagacttttcctcacccccaaagcaacaatgatcagcaaagagaaggatcattctttggccagactaaagtggaagaatgttttcatggtgaaaatcagtattcaaaatcaagcgagttcgagactcataatgtccaaatgggactggaggaagtacagaatataaatcgtagaaattccccttatagtcagaccatgaaatcaagtgcatgcaaaatacaggtttcttgttcaaacaatacacacctagtttcagagaataaagaacagactacacatcctgaactttttgcaggaaacaagacccaaaacttgcatcacatgcaatattttccaaataatgtgatcccaaagcaagatcttcttcacaggtgctttcaagaacaggagcagaagtcacaacaagcttcagttctacagggatataaaaatagaaaccaagatatgtctggtcaacaagctgcgcaacttgctcagcaaaggtacttgatacataaccatgcaaatgtttttcctgtgcctgaccagggaggaagtcacactcagacccctccccagaaggacactcaaaagcatgctgctctaaggtggcatctcttacagaagcaagaacagcagcaaacacagcaaccccaaactgagtcttgccatagtcagatgcacaggccaattaaggtggaacctggatgcaagccacatgcctgtatgcacacagcaccaccagaaaacaaaacatggaaaaaggtaactaagcaagagaatccacctgcaagctgtgataatgtgcagcaaaagagcatcattgagaccatggagcagcatctgaagcagtttcacgccaagtcgttatttgaccataaggctcttactctcaaatcacagaagcaagtaaaagttgaaatgtcagggccagtcacagttttgactagacaaaccactgctgcagaacttgatagccacaccccagctttagagcagcaaacaacttcttcagaaaagacaccaaccaaaagaacagctgcttctgttctcaataattttatagagtcaccttccaaattactagatactcctataaaaaatttattggatacacctgtcaagactcaatatgatttcccatcttgcagatgtgtagagcaaattattgaaaaagatgaaggtcctttttatacccatctaggagcaggtcctaatgtggcagctattagagaaatcatggaagaaaggtttggacagaagggtaaagctattaggattgaaagagtcatctatactggtaaagaaggcaaaagttctcagggatgtcctattgctaagtgggtggttcgcagaagcagcagtgaagagaagctactgtgtttggtgcgggagcgagctggccacacctgtgaggctgcagtgattgtgattctcatcctggtgtgggaaggaatcccgctgtctctggctgacaaactctactcggagcttaccgagacgctgaggaaatacggcacgctcaccaatcgccggtgtgccttgaatgaagagagaacttgcgcctgtcaggggctggatccagaaacctgtggtgcctccttctcttttggttgttcatggagcatgtactacaatggatgtaagtttgccagaagcaagatcccaaggaagtttaagctgcttggggatgacccaaaagaggaagagaaactggagtctcatttgcaaaacctgtccactcttatggcaccaacatataagaaacttgcacctgatgcatataataatcagattgaatatgaacacagagcaccagagtgccgtctgggtctgaaggaaggccgtccattctcaggggtcactgcatgtttggacttctgtgctcatgcccacagagacttgcacaacatgcagaatggcagcacattggtatgcactctcactagagaagacaatcgagaatttggaggaaaacctgaggatgagcagcttcacgttctgcctttatacaaagtctctgacgtggatgagtttgggagtgtggaagctcaggaggagaaaaaacggagtggtgccattcaggtactgagttcttttcggcgaaaagtcaggatgttagcagagccagtcaagacttgccgacaaaggaaactagaagccaagaaagctgcagctgaaaagctttcctccctggagaacagctcaaataaaaatgaaaaggaaaagtcagccccatcacgtacaaaacaaactgaaaacgcaagccaggctaaacagttggcagaacttttgcgactttcaggaccagtcatgcagcagtcccagcagccccagcctctacagaagcagccaccacagccccagcagcagcagagaccccagcagcagcagccacatcaccctcagacagagtctgtcaactcttattctgcttctggatccaccaatccatacatgagacggcccaatccagttagtccttatccaaactcttcacacacttcagatatctatggaagcaccagccctatgaacttctattccacctcatctcaagctgcaggttcatatttgaattcttctaatcccatgaacccttaccctgggcttttgaatcagaatacccaatatccatcatatcaatgcaatggaaacctatcagtggacaactgctccccatatctgggttcctattctccccagtctcagccgatggatctgtataggtatccaagccaagaccctctgtctaagctcagtctaccacccatccatacactttaccagccaaggtttggaaatagccagagttttacatctaaatacttaggttatggaaaccaaaatatgcagggagatggtttcagcagttgtaccattagaccaaatgtacatcatgtagggaaattgcctccttatcccactcatgagatggatggccacttcatgggagccacctctagattaccacccaatctgagcaatccaaacatggactataaaaatggtgaacatcattcaccttctcacataatccataactacagtgcagctccgggcatgttcaacagctctcttcatgccctgcatctccaaaacaaggagaatgacatgctttcccacacagctaatgggttatcaaagatgcttccagctcttaaccatgatagaactgcttgtgtccaaggaggcttacacaaattaagtgatgctaatggtcaggaaaagcagccattggcactagtccagggtgtggcttctggtgcagaggacaacgatgaggtctggtcagacagcgagcagagctttctggatcctgacattgggggagtggccgtggctccaactcatgggtcaattctcattgagtgtgcaaagcgtgagctgcatgccacaacccctttaaagaatcccaataggaatcaccccaccaggatctccctcgtcttttaccagcataagagcatgaatgagccaaaacatggcttggctctttgggaagccaaaatggctgaaaaagcccgtgagaaagaggaagagtgtgaaaagtatggcccagactatgtgcctcagaaatcccatggcaaaaaagtgaaacgggagcctgctgagccacatgaaacttcagagcccacttacctgcgtttcatcaagtctcttgccgaaaggaccatgtccgtgaccacagactccacagtaactacatctccatatgccttcactcgggtcacagggccttacaacagatatatatgatatcacccccttttgttggttacctcacttgaaaagaccacaaccaacctgtcagtagtatagttctcatgacgtgggcagtggggaaaggtcacagtattcatgacaaatgtggtgggaaaaacctcagctcaccagcaacaaaagaggttatcttaccatagcacttaattttcactggctcccaagtggtcacagatggcatctaggaaaagaccaaagcattctatgcaaaaagaaggtggggaagaaagtgttccgcaatttacatttttaaacactggttctattattggacgagatgatatgtaaatgtgatcccccccccccgcttacaactctacacatctgtgaccacttttaataatatcaagtttgcatagtcatggaacacaaatcaaacaagtactgtagtattacagtgacaggaatcttaaaataccatctggtgctgaatatatgatgtactgaaatactggaattatggctttttgaaatgcagtttttactgtaatcttaacttttatttatcaaaatagctacaggaaacatgaatagcaggaaaacactgaatttgtttggatgttctaagaaatggtgctaagaaaatggtgtctttaatagctaaaaatttaatgcctttatatcatcaagatgctatcagtgtactccagtgcccttgaataataggggtaccttttcattcaagtttttatcataattacctattcttacacaagcttagtttttaaaatgtggacattttaaaggcctctggattttgctcatccagtgaagtccttgtaggacaataaacgtatatatgtacatatatacacaaacatgtatatgtgcacacacatgtatatgtataaatattttaaatggtgttttagaagcactttgtctacctaagctttgacaacttgaacaatgctaaggtactgagatgtttaaaaaacaagtttactttcattttagaatgcaaagttgatttttttaaggaaacaaagaaagcttttaaaatatttttgcttttagccatgcatctgctgatgagcaattgtgtccatttttaacacagccagttaaatccaccatggggcttactggattcaagggaatacgttagtccacaaaacatgttttctggtgctcatctcacatgctatactgtaaaacagttttatacaaaattgtatgacaagttcattgctcaaaaatgtacagttttaagaattttctattaactgcaggtaataattagctgcatgctgcagactcaacaaagctagttcactgaagcctatgctattttatggatcataggctcttcagagaactgaatggcagtctgcctttgtgttgataattatgtacattgtgacgttgtcatttcttagcttaagtgtcctctttaacaagaggattgagcagactgatgcctgcataagatgaataaacagggttagttccatgtgaatctgtcagttaaaaagaaacaaaaacaggcagctggtttgctgtggtggttttaaatcattaatttgtataaagaagtgaaagagttgtatagtaaattaaattgtaaacaaaacttttttaatgcaatgctttagtattttagtactgtaaaaaaattaaatatatacatatatatatatatatatatatatatatatatgagtttgaagcagaattcacatcatgatggtgctactcagcctgctacaaatatatcataatgtgagctaagaattcattaaatgtttgagtgatgttcctacttgtcatatacctcaacactagtttggcaataggatattgaactgagagtgaaagcattgtgtaccatcatttttttccaagtccttttttttattgttaaaaaaaaaagcataccttttttcaatacttgatttcttagcaagtataacttgaacttcaacctttttgttctaaaaattcagggatatttcagctcatgctctccctatgccaacatgtcacctgtgtttatgtaaaattgttgtaggttaataaatatattctttgtcagggatttaacccttttattttgaatcccttctattttacttgtacatgtgctgatgtaactaaaactaattttgtaaatctgttggctctttttattgtaaagaaaagcattttaaaagtttgaggaatcttttgactgtttcaagcaggaaaaaaaaattacatgaaaatagaatgcactgagttgataaagggaaaaattgtaaggcaggagtttggcaagtggctgttggccagagacttacttgtaactctctaaatgaagtttttttgatcctgtaatcactgaaggtacatactccatgtggacttcccttaaacaggcaaacacctacaggtatggtgtgcaacagattgtacaattacattttggcctaaatacatttttgcttactagtatttaaaataaattcttaatcagaggaggcctttgggttttattggtcaaatctttgtaagctggcttttgtctttttaaaaaatttcttgaatttgtggttgtgtccaatttgcaaacatttccaaaaatgtttgctttgcttacaaaccacatgattttaatgttttttgtataccataatatctagccccaaacatttgattactacatgtgcattggtgattttgatcatccattcttaatatttgatttctgtgtcacctactgtcatttgttaaactgctggccaacaagaacaggaagtatagtttggggggttggggagagtttacataaggaagagaagaaattgagtggcatattgtaaatatcagatctataattgtaaatataaaacctgcctcagttagaatgaatggaaagcagatctacaatttgctaatataggaatatcaggttgactatatagccatacttgaaaatgcttctgagtggtgtcaactttacttgaatgaatttttcatcttgattgacgcacagtgatgtacagttcacttctgaagctagtggttaacttgtgtaggaaacttttgcagtttgacactaagataacttctgtgtgcatttttctatgcttttttaaaaactagtttcatttcattttcatgagatgtttggtttataagatctgaggatggttataaatactgtaagtattgtaatgttatgaatgcaggttatttgaaagctgtttattattatatcattcctgataatgctatgtgagtgtttttaataaaatttatatttatttaatgcactctaaaaaaaaaaaaaaaaaa

SEQ ID NO: 11 - protein, 1795 aa, Homo sapiens
MSQFQVPLAVQPDLPGLYDFPQRQVMVGSFPGSGLSMAGSESQLRGGGDGRKKRKRCGTCEPCRRLENCGACTSCTNRRTHQICKLRKCEVLKKKVGLLKEVEIKAGEGAGPWGQGAAVKTGSELSPVDGPVPGQMDSGPVYHGDSRQLSASGVPVNGAREPAGPSLLGTGGPWRVDQKPDWEAAPGPAHTARLEDAHDLVAFSAVAEAVSSYGALSTRLYETFNREMSREAGNNSRGPRPGPEGCSAGSEDLDTLQTALALARHGMKPPNCNCDGPECPDYLEWLEGKIKSVVMEGGEERPRLPGPLPPGEAGLPAPSTRPLLSSEVPQISPQEGLPLSQSALSIAKEKNISLQTAIAIEALTQLSSALPQPSHSTPQASCPLPEALSPPAPFRSPQSYLRAPSWPVVPPEEHSSFAPDSSAFPPATPRTEFPEAWGTDTPPATPRSSWPMPRPSPDPMAELEQLLGSASDYIQSVFKRPEALPTKPKVKVEAPSSSPAPAPSPVLQREAPTPSSEPDTHQKAQTALQQHLHHKRSLFLEQVHDTSFPAPSEPSAPGWWPPPSSPVPRLPDRPPKEKKKKLPTPAGGPVGTEKAAPGIKPSVRKPIQIKKSRPREAQPLFPPVRQIVLEGLRSPASQEVQAHPPAPLPASQGSAVPLPPEPSLALFAPSPSRDSLLPPTQEMRSPSPMTALQPGSTGPLPPADDKLEELIRQFEAEFGDSFGLPGPPSVPIQDPENQQTCLPAPESPFATRSPKQIKIESSGAVTVLSTTCFHSEEGGQEATPTKAENPLTPTLSGFLESPLKYLDTPTKSLLDTPAKRAQAEFPTCDCVEQIVEKDEGPYYTHLGSGPTVASIRELMEERYGEKGKAIRIEKVIYTGKEGKSSRGCPIAKWVIRRHTLEEKLLCLVRHRAGHHCQNAVIVILILAWEGIPRSLGDTLYQELTDTLRKYGNPTSRRCGLNDDRTCACQGKDPNTCGASFSFGCSWSMYFNGCKYARSKTPRKFRLAGDNPKEEEVLRKSFQDLATEVAPLYKRLAPQAYQNQVTNEEIAIDCRLGLKEGRPFAGVTACMDFCAHAHKDQHNLYNGCTVVCTLTKEDNRCVGKIPEDEQLHVLPLYKMANTDEFGSEENQNAKVGSGAIQVLTAFPREVRRLPEPAKSCRQRQLEARKAAAEKKKIQKEKLSTPEKIKQEALELAGITSDPGLSLKGGLSQQGLKPSLKVEPQNHFSSFKYSGNAVVESYSVLGNCRPSDPYSMNSVYSYHSYYAQPSLTSVNGFHSKYALPSFSYYGFPSSNPVFPSQFLGPGAWGHSGSSGSFEKKPDLHALHNSLSPAYGGAEFAELPSQAVPTDAHHPTPHHQQPAYPGPKEYLLPKAPLLHSVSRDPSPFAQSSNCYNRSIKQEPVDPLTQAEPVPRDAGKMGKTPLSEVSQNGGPSHLWGQYSGGPSMSPKRTNGVGGSWGVFSSGESPAIVPDKLSSFGASCLAPSHFTDGQWGLFPGEGQQAASHSGGRLRGKPWSPCKFGNSTSALAGPSLTEKPWALGAGDFNSALKGSPGFQDKLWNPMKGEEGRIPAAGASQLDRAWQSFGLPLGSSEKLFGALKSEEKLWDPFSLEEGPAEEPPSKGAVKEEKGGGGAEEEEEELWSDSEHNFLDENIGGVAVAPAHGSILIECARRELHATTPLKKPNRCHPTRISLVFYQHKNLNQPNHGLALWEAKMKQLAERARARQEEAARLGLGQQEAKLYGKKRKWGGTVVAEPQQKEKKGVVPTRQALAVPTDSAVTVSSYAYTKVTGPYSRWI

SEQ ID NO: 12 - DNA, 11405 bp, Homo sapiens
atgagccagtttcaggtgcccctggccgtccagccggacctgccaggcctttatgacttccctcagcgccaggtgatggtagggagcttcccggggtctgggctctccatggctgggagtgagtcccaactccgagggggtggagatggtcgaaagaaacggaaacggtgtggtacttgtgagccctgccggcggctggaaaactgtggcgcttgcactagctgtaccaaccgccgcacgcaccagatctgcaaactgcgaaaatgtgaggtgctgaagaaaaaagtagggcttctcaaggaggtggaaataaaggctggtgaaggagccgggccgtggggacaaggagcggctgtcaagacaggctcagagctcagcccagttgatggacctgttccaggtcagatggactcagggccagtgtaccatggggactcacggcagctaagcgcctcaggggtgccggtcaatggtgctagagagcccgctggacccagtctgctggggactgggggtccttggcgggtagaccaaaagcccgactgggaggctgccccaggcccagctcatactgctcgcctggaagatgcccacgatctggtggccttttcggctgtggccgaagctgtgtcctcttatggggcccttagcacccggctctatgaaaccttcaaccgtgagatgagtcgtgaggctgggaacaacagcaggggaccccggccagggcctgagggctgctctgctggcagcgaagaccttgacacactgcagacggccctggccctcgcgcggcatggtatgaaaccacccaactgcaactgcgatggcccagaatgccctgactacctcgagtggctggaggggaagatcaagtctgtggtcatggaaggaggggaggagcggcccaggctcccagggcctctgcctcctggtgaggccggcctcccagcaccaagcaccaggccactcctcagctcagaggtgccccagatctctccccaagagggcctgcccctgtcccagagtgccctgagcattgccaaggaaaaaaacatcagcttgcagaccgccattgccattgaggccctcacacagctctcctctgccctcccgcagccttctcattccaccccccaggcttcttgcccccttcctgaggccttgtcacctcctgcccctttcagatctccccagtcttacctccgggctccctcatggcctgtggttcctcctgaagagcactcatcttttgctcctgatagctctgccttccctccagcaactcctagaactgagttccctgaagcctggggcactgacacccctccagcaacgccccggagctcctggcccatgcctcgcccaagccccgatcccatggctgaactggagcagttgttgggcagcgccagtgattacatccagtcagtattcaagcggcctgaggccctgcctaccaagcccaaggtcaaggtggaggcaccctcttcctccccggccccggccccatcccctgtacttcagagggaggctcccacgccatcctcggagcccgacacccaccagaaggcccagaccgccctgcagcagcacctccaccacaagcgcagcctcttcctagaacaggtgcacgacacctccttccctgctccttcagagccttctgctcctggctggtggcccccaccaagttcacctgtcccacggcttccagacagaccacccaaggagaagaagaagaagctcccaacaccagctggaggtcccgtgggaacggagaaagctgcccctgggatcaagcccagtgtccgaaagcccattcagatcaagaagtccaggccccgggaagcacagcccctcttcccacctgtccgacagattgtcctggaagggcttaggtccccagcctcccaggaagtgcaggctcatccaccggcccctctgcctgcctcacagggctctgctgtgcccctgcccccagaaccttctcttgcgctatttgcacctagtccctccagggacagcctgctgccccctactcaggaaatgaggtcccccagccccatgacagccttgcagccaggctccactggccctcttccccctgccgatgacaagctggaagagctcatccggcagtttgaggctgaatttggagatagctttgggcttcccggccccccttctgtgcccattcaggaccccgagaaccagcaaacatgtctcccagcccctgagagcccctttgctacccgttcccccaagcaaatcaagattgagtcttcgggggctgtgactgtgctctcaaccacctgcttccattcagaggagggaggacaggaggccacacccaccaaggctgagaacccactcacacccaccctcagtggcttcttggagtcacctcttaagtacctggacacacccaccaagagtctgctggacacacctgccaagagagcccaggccgagttccccacctgcgattgcgtcgaacaaatagtggagaaagatgaaggtccatattatactcacttgggatctggccccacggtcgcctctatccgggaactcatggaggagcggtatggagagaaggggaaagccatccggatcgagaaggtcatctacacggggaaggagggaaagagctcccgcggttgccccattgcaaagtgggtgatccgcaggcacacgctggaggagaagctactctgcctggtgcggcaccgggcaggccaccactgccagaacgctgtgatcgtcatcctcatcctggcctgggagggcattccccgtagcctcggagacaccctctaccaggagctcaccgacaccctccggaagtatgggaaccccaccagccggagatgcggcctcaacgatgaccggacctgcgcttgccaaggcaaagaccccaacacctgtggtgcctccttctcctttggttgttcctggagcatgtacttcaacggctgcaagtatgctcggagcaagacacctcgcaagttccgcctcgcaggggacaatcccaaagaggaagaagtgctccggaagagtttccaggacctggccaccgaagtcgctcccctgtacaagcgactggcccctcaggcctatcagaaccaggtgaccaacgaggaaatagcgattgactgccgtctggggctgaaggaaggacggcccttcgcgggggtcacggcctgcatggacttctgtgcccacgcccacaaggaccagcataacctctacaatgggtgcaccgtggtctgcaccctgaccaaggaagacaatcgctgcgtgggcaagattcccgaggatgagcagctgcatgttctccccctgtacaagatggccaacacggatgagtttggtagcgaggagaaccagaatgcaaaggtgggcagcggagccatccaggtgctcaccgccttcccccgcgaggtccgacgcctgcccgagcctgccaagtcctgccgccagcggcagctggaagccagaaaggcagcagccgagaagaagaagattcagaaggagaagctgagcactccggagaagatcaagcaggaggccctggagctggcgggcattacgtcggacccaggcctgtctctgaagggtggattgtcccagcaaggcctgaagccctccctcaaggtggagccgcagaaccacttcagctccttcaagtacagcggcaacgcggtggtggagagctactcggtgctgggcaactgccggccctccgacccttacagcatgaacagcgtgtactcctaccactcctactatgcacagcccagcctgacctccgtcaatggcttccactccaagtacgctctcccgtcttttagctactatggctttccatccagcaaccccgtcttcccctctcagttcctgggtcctggtgcctgggggcacagtggcagcagtggcagttttgagaagaagccagacctccacgctctgcacaacagcctgagcccggcctacggtggtgctgagtttgccgagctgcccagccaggctgttcccacagacgcccaccaccccactcctcaccaccagcagcctgcgtacccaggccccaaggagtatctgcttcccaaggcccccctactccactcagtgtccagggacccctccccctttgcccagagctccaactgctacaacagatccatcaagcaagagccagtagacccgctgacccaggctgagcctgtgcccagagacgctggcaagatgggcaagacacctctgtccgaggtgtctcagaatggaggacccagtcacctttggggacagtactcaggaggcccaagcatgtcccccaagaggactaacggtgtgggtggcagctggggtgtgttctcgtctggggagagtcctgccatcgtccctgacaagctcagttcctttggggccagctgcctggccccttcccacttcacagatggccagtgggggctgttccccggtgaggggcagcaggcagcttcccactctggaggacggctgcgaggcaaaccgtggagcccctgcaagtttgggaacagcacctcggccttggctgggcccagcctgactgagaagccgtgggcgctgggggcaggggatttcaactcggccctgaaaggtagtcctgggttccaagacaagctgtggaaccccatgaaaggagaggagggcaggattccagccgcaggggccagccagctggacagggcctggcagtcctttggtctgcccctgggatccagcgagaagctgtttggggctctgaagtcagaggagaagctgtgggaccccttcagcctggaggaggggccggctgaggagccccccagcaagggagcggtgaaggaggagaagggcggtggtggtgcggaggaggaagaggaggagctgtggtcggacagtgaacacaacttcctggacgagaacatcggcggcgtggccgtggccccagcccacggctccatcctcatcgagtgtgcccggcgggagctgcacgccaccacgccgcttaagaagcccaaccgctgccaccccacccgcatctcgctggtcttctaccagcacaagaacctcaaccagcccaaccacgggctggccctctgggaagccaagatgaagcagctggcggagagggcacgggcacggcaggaggaggctgcccggctgggcctgggccagcaggaggccaagctctacgggaagaagcgcaagtgggggggcactgtggttgctgagccccagcagaaagagaagaagggggtcgtccccacccggcaggcactggctgtgcccacagactcggcggtcaccgtgtcctcctatgcctacacgaaggtcactggcccctacagccgctggatctaggtgccagggagccagcgtacctcagcgtcgggcctggcccgagctgtctctgtggtgcttttgccctcatacctgggggcgggttgggggtgcagaagtctttttatctctatatacatatatagatgcgcatatcatatatatgtatttatggtccaaacctcagaactgacccgcccctcccttacccccacttccccagcactttgaagaagaaactacggctgtcgggtgatttttccgtgatcttaatatttatatctccaagttgtcccccccccttgtctggggggtttttatttttattttctctttgtttttaaaactctatccttgtatatcacaataatggaaagaaagtttatagtatcctttcacaaaggagtagttttaaattccatttaaaatgtgtatttattggattttttaaaagcgacaatagtaatggtaaaggatgggcaggaaaggccagtagtgctcccccgcccagtctcgctgggtctggcgagccaagcccctcgggcgctggcgaggtcctcagccatctgcccctcgagagccaagcgcggacggtagccacccagttcatccctcccgacatacaccccttccctttggggaagggagcctcaggacagcttctgtcctctctgataggatgggagagtctgcagaaaaccatctggggtcccttttccagtccccggcttggagtcgaagggcagatgcaccccaggccagccccacgagatgctggcatagctttccccagaaaccaggttggaagtagatggcttcaagcttgctagtctccacactgaatcctctgtccgttatttatggagtcacacgatgtcatggttcactaggcagcacctcacgctggagctggagtgcgaggttcttaggggccgtgcccaccatgttgccaagccaatgcatgctgagctgaaggaatttgtcttagtggcagttttttaaaaaatgcccccaaagtctatgctgatactgaaaaagggctactgtatctttaaaaacaggaagttgaacccaagctgtgaaaagccagtggtgctctgtgcatggtgctgtgcggagcctggtgctgtagtgttgtgctgggactttcttgactcttgggcaggtcacatcctacaggagctcagcagaccagtgtaacaacagttaatgcatctatcctgatccctgaatttccacattggacaatggtgcatgcctcacacctgagcctgcttcctccatgctgtcattgggttcgggggcctacacttaacaattttaaagtgcaagagtcaaacattttcaacaggttgctataattttcctccctaattggtgccatttctccatttgatcattttctttttttcctttctcccctcttcatccactttaatatagctgttctgaaattctggtgcattcattcggttctttgaaatgagaatgtggtgcttaatttttgtgacgttgtcgagagaggttgggcctgatgggagcaacactcatcatcaccaagtcaaactttgttggagtgttggtttttcttgtgatattagcagaaatgatctcatgctagccatgtggatgtgtgtgtggtgaatggggggcttcatcaggacacacagaggggaatgtggccacacggtggatgaccaccaagccctgagatgaacaggtatttactgagcagttgtattcagatatgggtcttcatgaatcatgtttaacaatcagatgaccgctataggcaagttcctgagcttccgggtgccttgagtaagagctgagaaccggcctgctgggtgtttactgtatctgtttggaagcactggcggagggtcgttgtaagatgtcctgagcatttatgtggtctggttttaactgtaaatagtgaaagatttttttaagcacttttgcctagatttaaacagcaacttgaaaaaaaaagtatgttttaacatgtaattgtgggagaaattgtaaatagtagccgaatatttaatgtgctttgtctatcctccacttttaccatattctgtaaagttgcatttattttacaggacaaaaaaatgaaatattattgcttttgaaataaatacccaagagcttatcaggacttagaattattcagaactcagatttataggaaaacctctgaccttcagtttgacaagctaaaggaagcagagtctttaatgagcatgctaattttctagttttgaggaaaaattgggtcctttaaatgctattttgcttatcgcatcagtacttttatgcaggtctcatttgactccgtgcttaggtagatgcgggggtgccttgaaaacttcattttaaatgatcttaagcaagaaatacaatattttacgaaacatttggagaatgtgaccgtctgtatgacccgtggaagccccaggttggctgttggtttggaaggtcccgagtgtaacccaggtgattctgatacttggcatgtgtgaatcttcctgatgtatgttaaataaactcttcccctcatcaccctttggtaggaaagccattagatgaaaggagaaaccaatacaagctaaaagcatgcgacgtctgtcccccagcccaaacagccttggttcatcagtttctgcagtaggagataggctgctgagaggtgagtcaagaggcagtctccattggatgtccccactccccgcagaatggcgtttccagagttaggcggtgtggttgccgtgctcaagcccatgctgatttgtacactacatgtctaacctacctcaaatctcagtcattaaaattagcatgctttagacatatatttaaaaagtaactatgcacagctctttatcccccccttgctgctgaagctttcttaaagagaaaaatcaaatttttattttttactggcactatcattttttaagtcctaaagatgattaacagacatttttatcatgagaagaaaaataaagccattgcaactaaagaacctaacagcatgaccaagttcgaagagtcatattatagcaacggaaatcgatggcgtcttagtcatctccccagtgtgccctgtccacggacaccatccacgtgcagtgcaaacatttggttccttttctgctctgttttgttttccctgcctgttgcgtgcaagggaagtgcttgtaaagttctgtgctacgagatttttaaaataaaaatcgcttcgcagcaggttctcacaaaataactggtgctagctcaagaaatcatcatctgaccatcagaaatcttgactaaaggtgttgcatggatttgggggtctttcggtttttggttttgggtctggcttttagcagggccaatgtttcccacaccccggcttcatgggtactgctttgccttctcaccaaggtgacgatggtgtgcgtggaaagagatgataccccaccgccccctcttggtccttccaccagcctcttttgggaacagtagtttgcagagcaagggatttttaaagcgctaaagcaaggaaagaagtagcagagcttaactgctttgtaccacacagcagtagatgtgcaaggacggttgacaatgagtcgatgataacctaatttcattgagagaaacccagccagacttgcttctagaggtttaatcaccatgagatctcaaaccaaggcaaagctggtggaaaactatatgatatccctgacgtgcctcaaccagtatctctttccttttgttactgaagtgtgttttatggactaggaagcatttttatgaattgaaatagtctaaataaaatggtgctatggtgttttaatgtgactgtccctgatcctgtcttgctgaggtgctatcaacgttctgaaaccacaaccaaccaaaaacaaggtgggctccagtctcttggcttttttttttccctcccctcttttggtgctgtcttagacccgtttaccgtgctataatctgctctgagcagtgttgtgttgtgttgtattgttcttcccttggtggccaaacaaagcaagtcgagaaggcagctatctccctttctgtgatcgggagtgggcctgcctggcttggcaggtgctttttggttccacacctgtcttctcaggcttgatgtgaaagaaagggcgaagggttttttgagtttttgtttttgaggaaggggagttgggtacttctgcctctcctagcatgataggcattctcatagccagggacagattttctcctgcagcccagggtgctaagcagacatctctgggagtcccaagggcacaccaagggagaccagatggatctccttcctcccctggcactggctgggaccatggtgggcaggggcttcattctctgacccagcgttgcttctgcctctcattggtaaccccttatgttcggactaaaggaaggagctttctttgctcactcgatgccactgaggctgctttttagttggtgctaacctaaatttcttcttgggtccacagaagttgatgttttaaaaactcaccaggaagctccattttgtgtcatccactgtcacaataatttttttaaatacctcaaaaacaggacatcatgacaacttcagtaaagtagattccatgagggtctgatacctgcaggttgtccgtctgatgacatacttgaccttgaaaaatctggggtcattttgtttttcattcttcagcagttaagatagcggaacgccgaaaggaaggagcgtagttggctgtatttcatgtttaagttttgcttttgaataaaatgtgaatttcctatgcccatctcattgagctttctcagtcattgttgctgtcatttgaaatgactccctcaaaacctagttttattagccagctgcctctgctgtagtacatggccaacttcaacataccctggaccaaaacatttttgaggtgcatacccccaacataagttacacagtcccacatccaggtgcacagagtgcgagtgcactccgcgagtgcggggggaggggcggccccctctggtgctcccagcccttcctcctgcagagctgcaggcaagagcagagcaataggcttctcccctgagcagagaccgcagcacagaaatgcaaggtctaaagttgctttttgcctaagaatcagcgagcgatttggcctacttcctcattggcttctattctgatatcagggatgctttttgtagtggtattgtttgctccctcttcgcgttttgactacccgtcattcaggggtaactcatcactcttcacacggggatttaaattaagaaactaattggctcatgtgaacattccaaattttcttggtttcaatacccttttttttcttttgaggggaaaagaggggagaaaaacaggagtgatgtcatttctttttcatgtattccaattaaagaaacaagggcaggtcgtataatggcatattaatacattagacttaatctagaacccctgtagctttttgatgtgttttatttcttatctctttgaattcctgtttggttacttggcttccaatggaggtgaacttaacaaccatacttgaatattccgtcttgactttgtaaactgtggctacttgaaatgaagtttatctggggttgatggatgaatggtagatttttgcaatgtctcaaggcaataggatgtgtattaaactgtagatattcttagtacagtaaatttatgctgataattttattttgtataatttttacctttttgttaatattttttccttccactttattggtttgcctcctgagctacccctccttaccctcccttctccctcagtgtttcagtaaatttaatttagggtgcctagaaattgcaagtatgtatcctttttgatttgtattttattataatttacacaaacaactgggtttgtgaactgtattactcctggtatctttaaaatattgtgggtgttttaataaattttatatttattttttgcactcaaaaaaaaaaaaaaaaaaaa

SEQ ID NO: 13 - protein, 4 aa, Artificial sequence
  linker peptide sequence
GGGS

SEQ ID NO: 14 - protein, 7 aa, Artificial sequence
  nuclear localization signal
PKKKRKV

SEQ ID NO: 15 - DNA, 6375 bp, Artificial sequence
  fusion protein (HA epitope,dCAS9, NLS, LINKER,      TET1 catalytic domain)
atgtacccatacgatgttccagattacgcttcgccgaagaaaaagcgcaaggtcgaagcgtccgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgccatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacagccccaagaagaagagaaaggtggaggccagcgggccggccggaggcggtggaagcctgcccacctgcagctgtcttgatcgagttatacaaaaagacaaaggcccatattatacacaccttggggcaggaccaagtgttgctgctgtcagggaaatcatggagaataggtatggtcaaaaaggaaacgcaataaggatagaaatagtagtgtacaccggtaaagaagggaaaagctctcatgggtgtccaattgctaagtgggttttaagaagaagcagtgatgaagaaaaagttctttgtttggtccggcagcgtacaggccaccactgtccaactgctgtgatggtggtgctcatcatggtgtgggatggcatccctcttccaatggccgaccggctatacacagagctcacagagaatctaaagtcatacaatgggcaccctaccgacagaagatgcaccctcaatgaaaatcgtacctgtacatgtcaaggaattgatccagagacttgtggagcttcattctcttttggctgttcatggagtatgtactttaatggctgtaagtttggtagaagcccaagccccagaagatttagaattgatccaagctctcccttacatgaaaaaaaccttgaagataacttacagagtttggctacacgattagctccaatttataagcagtatgctccagtagcttaccaaaatcaggtggaatatgaaaatgttgcccgagaatgtcggcttggcagcaaggaaggtcgacccttctctggggtcactgcttgcctggacttctgtgctcatccccacagggacattcacaacatgaataatggaagcactgtggtttgtaccttaactcgagaagataaccgctctttgggtgttattcctcaagatgagcagctccatgtgctacctctttataagctttcagacacagatgagtttggctccaaggaaggaatggaagccaagatcaaatctggggccatcgaggtcctggcaccccgccgcaaaaaaagaacgtgtttcactcagcctgttccccgttctggaaagaagagggctgcgatgatgacagaggttcttgcacataagataagggcagtggaaaagaaacctattccccgaatcaagcggaagaataactcaacaacaacaaacaacagtaagccttcgtcactgccaaccttagggagtaacactgagaccgtgcaacctgaagtaaaaagtgaaaccgaaccccattttatcttaaaaagttcagacaacactaaaacttattcgctgatgccatccgctcctcacccagtgaaagaggcatctccaggcttctcctggtccccgaagactgcttcagccacaccagctccactgaagaatgacgcaacagcctcatgcgggttttcagaaagaagcagcactccccactgtacgatgccttcgggaagactcagtggtgccaatgctgcagctgctgatggccctggcatttcacagcttggcgaagtggctcctctccccaccctgtctgctcctgtgatggagcccctcattaattctgagccttccactggtgtgactgagccgctaacgcctcatcagccaaaccaccagccctccttcctcacctctcctcaagaccttgcctcttctccaatggaagaagatgagcagcattctgaagcagatgagcctccatcagacgaacccctatctgatgaccccctgtcacctgctgaggagaaattgccccacattgatgagtattggtcagacagtgagcacatctttttggatgcaaatattggtggggtggccatcgcacctgctcacggctcggttttgattgagtgtgcccggcgagagctgcacgctaccactcctgttgagcaccccaaccgtaatcatccaacccgcctctcccttgtcttttaccagcacaaaaacctaaataagccccaacatggttttgaactaaacaagattaagtttgaggctaaagaagctaagaataagaaaatgaaggcctcagagcaaaaagaccaggcagctaatgaaggtccagaacagtcctctgaagtaaatgaattgaaccaaattccttctcataaagcattaacattaacccatgacaatgttgtcaccgtgtccccttatgctctcacacacgttgcggggccctataaccattgggtc

SEQ ID NO: 16 - DNA, 24 bp, Artificial sequence
  sgRNA
caccgggccggcgcctgagaaaac

SEQ ID NO: 17 - DNA, 24 bp, Artificial sequence
  sgRNA
aaacgttttctcaggcgccggccc

SEQ ID NO: 18 - DNA, 24 bp, Artificial sequence
  sgRNA
caccgcgcctgagtcagagaagcc

SEQ ID NO: 19 - DNA, 24 bp, Artificial sequence
  sgRNA
aaacggcttctctgactcaggcgc

SEQ ID NO: 20 - DNA, 24 bp, Artificial sequence
  sgRNA
caccgaatatttggaatcacagct

SEQ ID NO: 21 - DNA, 24 bp, Artificial sequence
  sgRNA
aaacagctgtgattccaaatattc

SEQ ID NO: 22 - DNA, 24 bp, Artificial sequence
  sgRNA
caccgatttgtgtaataagaaaat

SEQ ID NO: 23 - DNA, 24 bp, Artificial sequence
  sgRNA
aaacattttcttattacacaaatc

SEQ ID NO: 24 - DNA, 24 bp, Artificial sequence
  sgRNA
caccgtacgtaaatacacttgcaa

SEQ ID NO: 25 - DNA, 24 bp, Artificial sequence
  sgRNA
aaacttgcaagtgtatttacgtac

SEQ ID NO: 26 - DNA, 24 bp, Artificial sequence
  sgRNA
caccggacttcttctcccgcctct

SEQ ID NO: 27 - DNA, 24 bp, Artificial sequence
  sgRNA
aaacagaggcgggagaagaagtcc

SEQ ID NO: 28 - DNA, 24 bp, Artificial sequence
  sgRNA
caccggggcacctgcaccgacctc

SEQ ID NO: 29 - DNA, 24 bp, Artificial sequence
  sgRNA
aaacgaggtcggtgcaggtgcccc

SEQ ID NO: 30 - DNA, 24 bp, Artificial sequence
  sgRNA
caccggctagcaccagcgctctgt

SEQ ID NO: 31 - DNA, 24 bp, Artificial sequence
  sgRNA
aaacacagagcgctggtgctagcc

SEQ ID NO: 32 - DNA, 45 bp, Artificial sequence
  Single strand DNA oligonucleotide
agtggccggccggaggcggtggaagcctgcccacctgcagctgtc

SEQ ID NO: 33 - DNA, 24 bp, Artificial sequence
  Single strand DNA oligonucleotide
tcgaattctcagacccaatggtta

SEQ ID NO: 34 - DNA, 22 bp, Artificial sequence
  Single strand DNA oligonucleotide
cgagtattacccctatctcagc

SEQ ID NO: 35 - DNA, 18 bp, Artificial sequence
  Single strand DNA oligonucleotide
ctggtggccaagactggg

SEQ ID NO: 36 - DNA, 20 bp, Artificial sequence
  Single strand DNA oligonucleotide
gctctctgctcctcctgttc

SEQ ID NO: 37 - DNA, 19 bp, Artificial sequence
  Single strand DNA oligonucleotide
cgttgactccgaccttcac

SEQ ID NO: 38 - DNA, 20 bp, Artificial sequence
  Single strand DNA oligonucleotide
caagggcacctttgccacac

SEQ ID NO: 39 - DNA, 20 bp, Artificial sequence
  Single strand DNA oligonucleotide
tttgccaaagtgatgggcca

SEQ ID NO: 40 - DNA, 20 bp, Artificial sequence
  Single strand DNA oligonucleotide
ctacctccaccatgccaagt

SEQ ID NO: 41 - DNA, 20 bp, Artificial sequence
  Single strand DNA oligonucleotide
gcagtagctgcgctgataga

SEQ ID NO: 42 - DNA, 21 bp, Artificial sequence
  Single strand DNA oligonucleotide
tgacactggcaaaacaatgca

SEQ ID NO: 43 - DNA, 21 bp, Artificial sequence
  Single strand DNA oligonucleotide
ggtccttttcaccagcaagct

SEQ ID NO: 44 - DNA, 18 bp, Artificial sequence
  Single strand DNA oligonucleotide
cttgggtctggggtctgg

SEQ ID NO: 45 - DNA, 20 bp, Artificial sequence
  Single strand DNA oligonucleotide
ctgtggtaatgggctgttgg

SEQ ID NO: 46 - DNA, 20 bp, Artificial sequence
  Single strand DNA oligonucleotide
ccatcactgctccacaatca

SEQ ID NO: 47 - DNA, 20 bp, Artificial sequence
  Single strand DNA oligonucleotide
actccgagtggctcctagtg

SEQ ID NO: 48 - DNA, 62 bp, Artificial sequence
  Single strand DNA oligonucleotide
tcgtcggcagcgtcagatgtgtataagagacagggaattatgttgggttatatgaaatttaa

SEQ ID NO: 49 - DNA, 59 bp, Artificial sequence
  Single strand DNA oligonucleotide
gtctcgtgggctcggagatgtgtataagagacagtctaccccctcctcctaaataataa

SEQ ID NO: 50 - DNA, 58 bp, Artificial sequence
  Single strand DNA oligonucleotide
tcgtcggcagcgtcagatgtgtataagagacagtttttttatggaatagagggtgtag

SEQ ID NO: 51 - DNA, 64 bp, Artificial sequence
  Single strand DNA oligonucleotide
gtctcgtgggctcggagatgtgtataagagacagacttctacattctaattatcatatccttct

SEQ ID NO: 52 - DNA, 59 bp, Artificial sequence
  Single strand DNA oligonucleotide
gtctcgtgggctcggagatgtgtataagagacagggattttaaatttttagtttttttt

SEQ ID NO: 53 - DNA, 63 bp, Artificial sequence
  Single strand DNA oligonucleotide
tcgtcggcagcgtcagatgtgtataagagacagacttttaatacatcaacttcttatttatat

SEQ ID NO: 54 - DNA, 59 bp, Artificial sequence
  Single strand DNA oligonucleotide
tcgtcggcagcgtcagatgtgtataagagacagggtgtgagtggaataatttaagtttg

SEQ ID NO: 55 - DNA, 61 bp, Artificial sequence
  Single strand DNA oligonucleotide
gtctcgtgggctcggagatgtgtataagagacagcatccaccctctttataaccattataa

SEQ ID NO: 56 - DNA, 60 bp, Artificial sequence
  Single strand DNA oligonucleotide
tcgtcggcagcgtcagatgtgtataagagacaggggttgtagttgtttttgtttttatat

SEQ ID NO: 57 - DNA, 59 bp, Artificial sequence
  Single strand DNA oligonucleotide
gtctcgtgggctcggagatgtgtataagagacagactaaacatccccctaaaacctaac

SEQ ID NO: 58 - DNA, 24 bp, Artificial sequence
  Single strand DNA oligonucleotide
aagaggaaagaggtagtaagagtt

SEQ ID NO: 59 - DNA, 22 bp, Artificial sequence
  Single strand DNA oligonucleotide
  misc_feature (1)..(1): 5′ biotin
aatcactcactttacccctatc

SEQ ID NO: 60 - DNA, 19 bp, Artificial sequence
  Single strand DNA oligonucleotide
aagaggtagtaagagtttt

SEQ ID NO: 61 - DNA, 97 bp, Artificial Sequence
  exemplary gRNA that can be used for the present      invention
  misc_feature (1)..(20): n is a, c, g, t or u
nnnnnnnnnnnnnnnnnnnnguuuuagacuagaaauagcuuaaaauaggcuaguaguccguuaucaacuugaaaaagugcaccgagucggugcuuuu